(19) United States
(12) Patent Application Publication
(10) Pub. No.: US 2026/0096732 A1
(43) Pub. Date: Apr. 9, 2026

(54) Cardiac Monitoring System with Sleep and Activity Correlation

(71) Applicant: iRhythm Technologies, Inc., San Francisco, CA (US)

(72) Inventors: Yuriko Tamura, San Mateo, CA (US); Elaine Yuiyi Yu, Walnut Creek, CA (US); Jasmine Yu Hu, Shoreline, WA (US); Andrew David Gilbert, Vancouver (CA)

(73) Assignee: iRhythm Technologies, Inc., San Francisco, CA (US)

(21) Appl. No.: 19/347,508

(22) Filed: Oct. 1, 2025

Related U.S. Application Data
(60) Provisional application No. 63/703,761, filed on Oct. 4, 2024.
(60) Provisional application No. 63/703,806, filed on Oct. 4, 2024.
(60) Provisional application No. 63/703,833, filed on Oct. 4, 2024.

(51) Int. Cl. A61B 5/0205; A61B 5/00; A61B 5/11

(52) U.S. Cl. CPC A61B 5/0205; A61B 5/1116; A61B 5/1118; A61B 5/4809; A61B 5/4812; A61B 5/6832; A61B 5/7267; A61B 5/7275; A61B 5/742; A61B 2562/0219

(57) ABSTRACT

A cardiac monitoring system with sleep and activity correlation is described. In one or more implementations, measurements of a user generated by a wearable monitoring device during an observation period are obtained, the measurements including accelerometer data and electrical potential measurements. Sleep periods and wake periods are detected based on the accelerometer data. Sleep stage classifications and activity level classification are generated based on the accelerometer data during the detected sleep periods. The electrical potential measurements are processed using a machine learning model trained to correlate patterns in the electrical potential measurements to cardiac rhythm classifications. Arrhythmia correlations may be generated and output based on the sleep stage classifications and the activity level classifications and concurrent cardiac rhythm classifications. The arrhythmia correlations may describe temporal relationships between detected cardiac arrhythmias and specific sleep stages of the sleep stage classifications and may be output in a health report.

### RELATED APPLICATIONS

This application claims priority to U.S. Provisional Application No. 63/703,761, filed Oct. 4, 2024, and titled “Arrhythmia Detection and Correlation with Analysis Triggering,” to U.S. Provisional Application No. 63/703,806, filed Oct. 4, 2024, and titled “Ambulatory Cardiac Stress Test,” and to U.S. Provisional Application No. 63/703,833, filed Oct. 4, 2024, and titled “Sleep and Activity Prediction with Constrained Conditions,” which are hereby incorporated by reference in their entireties.

### BACKGROUND

Cardiac monitoring devices are commonly used to detect and analyze heart rhythm abnormalities over extended periods. Traditional electrocardiogram (ECG) monitoring systems collect continuous cardiac data but often lack contextual information about patient activities and physiological states during data collection. Existing wearable cardiac monitors typically operate with high-frequency data acquisition across multiple sensors, which can rapidly deplete battery resources and consume substantial memory storage capacity. Additionally, conventional cardiac monitoring approaches may process and analyze rhythm data in isolation, without correlating detected arrhythmias to concurrent patient behaviors such as sleep patterns, physical activity levels, or body positioning. Furthermore, continuous high-power sensor operation in wearable devices can compromise long-term patient compliance due to needing frequent device charging or battery replacement and reduced wearing comfort, which may affect the quality and completeness of collected cardiac data.

### BRIEF DESCRIPTION OF THE DRAWINGS

FIG. 1 is a block diagram of a non-limiting example of an environment that is operable to employ cardiac monitoring with sleep and activity correlation as described herein.

FIG. 2 depicts a non-limiting example of a monitoring device.

FIG. 3 depicts a non-limiting system in an example implementation of cardiac monitoring with sleep and activity correlation showing operation of the prediction system of FIG. 1 in more detail.

FIG. 4 depicts a monitoring system in a non-limiting example of cardiac monitoring with sleep stage and activity correlation in which the monitoring system generates a comprehensive health report for output.

FIGS. 5A and 5B show a non-limiting example of a user interface for displaying patient cardiac monitoring data with sleep and activity correlation.

FIG. 6 shows an example of a comprehensive activity-based cardiac assessment interface.

FIG. 7 shows an example of a multi-level activity classification display that provides comprehensive analysis of cardiac activity across different exercise intensities.

FIG. 8 shows an example of an ambulatory cardiac stress test interface that facilitates monitoring and analysis of cardiac activity during extended wear periods.

FIG. 9 depicts a flow diagram depicting an algorithm as a step-by-step procedure in an example implementation that is performable by a processing device to correlate cardiac monitoring with sleep and activity data based on measurements from a wearable monitoring device.

FIG. 10 depicts a flow diagram depicting an algorithm as a step-by-step procedure in an example implementation that is performable by a processing device to perform cardiac monitoring with activity level classification and arrhythmia correlation.

FIG. 11 depicts a flow diagram depicting an algorithm as a step-by-step procedure in which ambulatory cardiac stress testing is performed using a wearable monitoring device.

FIG. 12 depicts a flow diagram depicting an algorithm as a step-by-step procedure in which sleep/activity triggering of analysis and data acquisition is performed.

### DETAILED DESCRIPTION

Conventional cardiac monitoring systems typically collect continuous electrocardiogram (ECG) data using high-frequency sampling rates (e.g., 25-100 Hz) and multiple sensors to detect heart rhythm abnormalities over extended periods. These conventional approaches often operate in isolation from contextual information about patient activities, sleep patterns, and physiological states during data collection. Traditional ECG monitoring methods lack the ability to correlate detected arrhythmias with concurrent patient behaviors such as sleep stages, physical activity levels, or body positioning, which may hinder clinical assessments due to the lack of information about the circumstances surrounding detected cardiac events. Additionally, conventional wearable cardiac monitors typically rely on continuous high-power sensor operation across multiple channels, which may rapidly deplete battery resources and consume substantial memory storage capacity. This high power consumption may compromise long-term patient compliance due to frequent device charging and/or reduced wearing comfort, which may affect the quality and completeness of collected cardiac data. Furthermore, conventional systems may struggle to provide comprehensive physiological insights that combine cardiac monitoring with sleep and activity analysis, limiting their ability to identify relationships between cardiac events and daily life patterns.

Accordingly, techniques and systems for cardiac monitoring with sleep and activity correlation are described that address these limitations by providing comprehensive cardiac analyses that correlate detected arrhythmias with concurrent sleep stages and/or activity levels while operating efficiently with low-power sensor configurations. In one or more implementations, a wearable monitoring device obtains measurements of a user during an observation period, with the measurements including accelerometer data and electrical potential measurements. The accelerometer data is collected at a sampling rate of less than 5 Hertz (Hz) to conserve battery life and reduce power consumption while maintaining accurate detection capabilities for sleep and activity patterns. Sleep periods and wake periods are detected based on patterns in the accelerometer data. By way of example, an analysis system may analyze body angle calculations and movement patterns to distinguish between different physiological states. Sleep stage classifications are generated based on the accelerometer data during the detected sleep periods, categorizing sleep phases as light sleep, deep sleep, rapid eye movement (REM) sleep, and/or non-REM sleep to provide detailed temporal mapping of sleep architecture. The system further generates activity level classifications based on detected steps during wake periods, categorizing physical activity as sedentary, light, moderate, or vigorous based on movement equivalent to at least a threshold speed as determined from the accelerometer data.

The system processes the electrical potential measurements using at least one machine learning model trained to correlate patterns in the electrical potential measurements to cardiac rhythm classifications, enabling identification of various arrhythmia types and cardiac events throughout the observation period. Arrhythmia correlations are generated based on the sleep stage classifications and/or activity level classifications and concurrent cardiac rhythm classifications, with these correlations describing temporal relationships between detected cardiac arrhythmias and specific sleep stages and/or activity levels. In one or more implementations, an odds ratio analysis is generated based on the arrhythmia correlations, quantifying the likelihood of specific arrhythmias occurring during sleep periods versus wake periods and calculating statistical associations between specific arrhythmias and sleep periods across user populations.

In one or more implementations, the system identifies exercise periods performed by the user during the observation period based on the accelerometer data and generates arrhythmia correlations for periods before, during, and after the exercise periods based on the concurrent cardiac rhythm classifications. The system detects chronotropic incompetence by comparing expected heart rate responses with actual heart rate responses during activity events, identifying cases where cardiac output fails to increase appropriately during physical exertion.

In additional implementations, the system performs ambulatory cardiac stress testing by correlating exercise-related activity patterns with concurrent cardiac rhythm changes. The system may detect exercise periods from the accelerometer data and analyze concurrent cardiac responses from the electrical potential data to identify exercise-induced arrhythmias and cardiac recovery patterns that may indicate underlying coronary heart disease or other cardiovascular conditions. The ambulatory stress testing capabilities may include detection of ST segment depression, ST segment elevation, T wave inversion, T wave flattening, and pseudonormalization of T waves by comparing exercise and recovery electrocardiogram data to resting baseline measurements. Body position may be determined from accelerometer data during post-exercise recovery periods, with the system identifying when patients transition to supine positions that may precipitate ischemic abnormalities not visible during exercise. Instructions may be provided to the user to facilitate collection of data that will facilitate the ambulatory cardiac stress testing.

The system may implement sleep/activity triggering of analysis and data acquisition, where different sleep stages and/or activity levels trigger adaptive monitoring strategies to reduce battery usage while ensuring comprehensive data capture during physiologically relevant periods. When sleep is detected by accelerometer analysis, for example, the system may activate LED-based photoplethysmography for oxygen saturation measurement, which may be used for sleep apnea screening and diagnosis put is more battery intensive. The LED-based photoplethysmography may deactivate when the user is no longer asleep to conserve battery resources. When activity is detected, the system may activate additional sensors such as a gyroscope to provide additional data that may be used to distinguish activity type or intensity, while such sensors may remain inactive during periods of inactivity to conserve power. The selective sensor activation approach may enable the system to provide comprehensive physiological monitoring while extending battery life and improving long-term patient compliance during extended observation periods.

The described techniques provide substantial advantages over conventional approaches by enabling comprehensive cardiac monitoring that correlates detected arrhythmias with concurrent sleep and activity states while operating with low-power sensor configurations that extend battery life and improve patient compliance. The integration of accelerometer-based sleep and activity detection with concurrent cardiac monitoring provides comprehensive physiological insights that are not achievable through separate analysis of individual sensor modalities, enabling healthcare providers to understand cardiac events within the context of daily life patterns and sleep architecture. The sensor operation at low sampling frequencies while maintaining high accuracy in both sleep stage detection and activity classification reduces hardware complexity and power consumption compared to conventional multi-sensor systems. Moreover, the correlation between cardiac events and sleep stages and/or activity levels enables healthcare providers to make informed clinical decisions without relying solely on patient recollection, particularly in cases where specific sleep stages or activity intensities may trigger arrhythmic episodes. Further discussion of these and other examples and advantages are included in the following sections and shown using corresponding figures.

In some aspects, the techniques described herein relate to a method implemented by a processing device, the method including: obtaining measurements of a user generated by a wearable monitoring device during an observation period, the measurements including accelerometer data and electrical potential measurements; detecting sleep periods and wake periods based on the accelerometer data; generating sleep stage classifications based on the accelerometer data during the detected sleep periods; processing the electrical potential measurements using a machine learning model trained to correlate patterns in the electrical potential measurements to cardiac rhythm classifications; generate arrhythmia correlations based on the sleep stage classifications and concurrent cardiac rhythm classifications, the arrhythmia correlations describing temporal relationships between detected cardiac arrhythmias and specific sleep stages of the sleep stage classifications; and outputting a health report including the arrhythmia correlations.

In some aspects, the techniques described herein relate to a method, wherein the accelerometer data is collected at a sampling rate of less than 5 Hertz.

In some aspects, the techniques described herein relate to a method, wherein detecting the sleep periods and the wake periods based on the accelerometer data includes: detecting steps as peaks above a threshold in the accelerometer data; calculating body angle measurements from the accelerometer data to determine body positioning; and distinguishing between the sleep periods and the wake periods based on the detected steps and the body angle measurements.

In some aspects, the techniques described herein relate to a method, wherein generating the sleep stage classifications includes categorizing the sleep periods as light sleep, deep sleep, rapid eye movement (REM) sleep, and non-REM sleep.

In some aspects, the techniques described herein relate to a method, further including: indicating activity events during the wake periods based on the accelerometer data obtained during the wake periods; generating activity level classifications of the activity events based on an intensity of physical activity performed; and generating additional arrhythmia correlations based on the activity level classifications and corresponding concurrent cardiac rhythm classifications, the additional arrhythmia correlations describing temporal relationships between the detected cardiac arrhythmias and specific activity levels of the activity level classifications.

In some aspects, the techniques described herein relate to a method, wherein the activity level classifications categorize the physical activity as light, moderate, or vigorous based on movement equivalent to at least a threshold speed, as determined based on the accelerometer data.

In some aspects, the techniques described herein relate to a method, further including: detecting chronotropic incompetence based on an expected heart rate response relative to an actual heart rate response during the physical activity.

In some aspects, the techniques described herein relate to a method, further including generating an odds ratio analysis based on the arrhythmia correlations, the odds ratio analysis quantifying a likelihood of specific arrhythmias occurring during the sleep periods versus the wake periods.

In some aspects, the techniques described herein relate to a method, wherein the odds ratio analysis calculates statistical associations between the specific arrhythmias and the sleep periods across a user population.

In some aspects, the techniques described herein relate to a method, wherein the health report includes a visualization of the arrhythmia correlations with the sleep periods and the wake periods.

In some aspects, the techniques described herein relate to a processing device, including: one or more processors; and memory having stored computer-readable instructions that are executable by the one or more processors to perform operations including: obtaining measurements of a user generated by a wearable monitoring device during an observation period, the measurements including accelerometer data and electrical potential measurements; detecting sleep periods and wake periods based on the accelerometer data; generating sleep stage classifications based on the accelerometer data during the sleep periods; generating activity level classifications based on detected steps during the wake periods; processing the electrical potential measurements using a machine learning model trained to correlate patterns in the electrical potential measurements to cardiac rhythm classifications; correlating the sleep stage classifications and the activity level classifications with concurrent cardiac rhythm classifications to generate arrhythmia correlations; generating a cardiac wellness prediction based on the arrhythmia correlations; and outputting a health report including the cardiac wellness prediction.

In some aspects, the techniques described herein relate to a processing device, wherein the accelerometer data is collected at a sampling rate of less than 5 Hertz.

In some aspects, the techniques described herein relate to a processing device, wherein generating the activity level classifications includes categorizing physical activity as sedentary, light, moderate, or vigorous based on movement speed, as determined based on the accelerometer data.

In some aspects, the techniques described herein relate to a processing device, wherein the operations further include detecting chronotropic incompetence by comparing an expected heart rate response and an actual heart rate response of the user during activity events within the wake periods.

In some aspects, the techniques described herein relate to a processing device, wherein the operations further include: determining a heart rate of the user over time based on the electrical potential measurements; generating time series plots of the heart rate during detected the detected activity events; stratifying the time series plots based on the activity level classifications; and outputting the stratified time series plots as a part of the health report.

In some aspects, the techniques described herein relate to a system including: a wearable monitoring device that is wearable by a user to detect one or more measurements of the user during an observation period, the one or more measurements including accelerometer measurements and electrical potential measurements of a heart of the user; and a computing device configured to: receive the one or more measurements from the wearable monitoring device; identify exercise periods performed by the user during the observation period based on the accelerometer measurements; process the electrical potential measurements using a machine learning model trained to correlate patterns in the electrical potential measurements to cardiac rhythm classifications; generate arrhythmia correlations for periods before, during, and after the exercise periods based on concurrent cardiac rhythm classifications; and generate a cardiac wellness prediction based on the arrhythmia correlations.

In some aspects, the techniques described herein relate to a system, wherein the accelerometer measurements are collected at a sampling rate of less than 5 Hertz, and wherein the observation period is between 7 and 14 days.

In some aspects, the techniques described herein relate to a system, wherein the computing device is further configured to: generate an expected heart rate response during a given exercise period based on user population data; generate an actual heart rate response of the user during the given exercise period based on the electrical potential measurements obtained during the given exercise period; and indicate chronotropic incompetence based on the actual heart rate response deviating from the expected heart rate response by at least a threshold amount.

In some aspects, the techniques described herein relate to a system, wherein to identify the exercise periods based on the accelerometer measurements, the computing device is further configured to: detect steps as peaks in the accelerometer measurements above a measurement threshold; and identify the exercise periods based on the detected steps exceeding a step count threshold during a predetermined time epoch.

In some aspects, the techniques described herein relate to a system, wherein the computing device is further configured to: detect sleep periods of the user based on the accelerometer measurements; generate sleep stage classifications based on the accelerometer measurements during the detected sleep periods; and generate additional arrhythmia correlations based on the sleep stage classifications and the concurrent cardiac rhythm classifications.

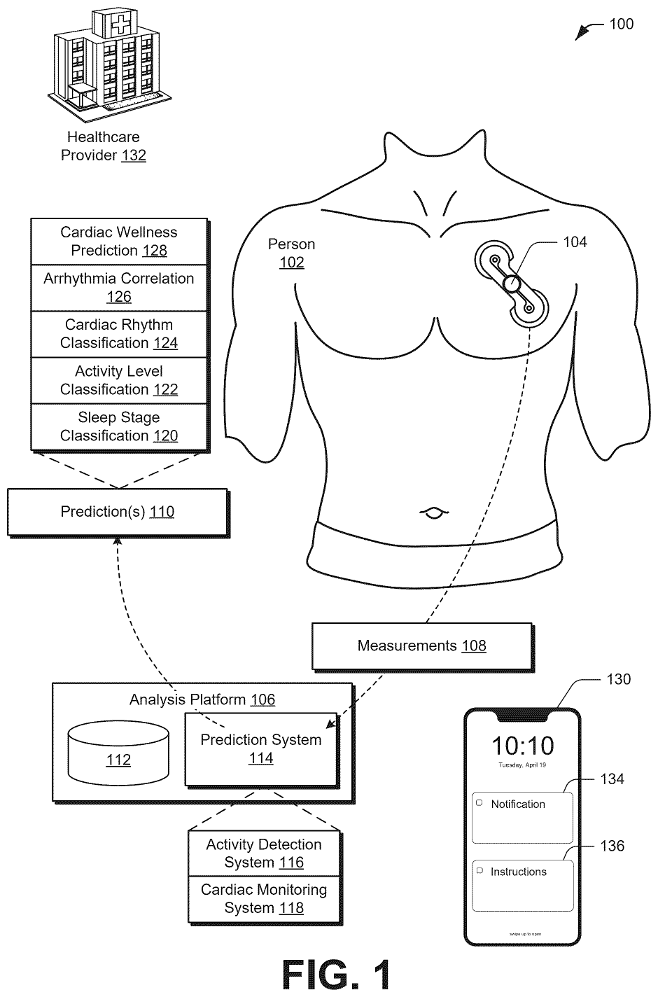

FIG. 1 is a block diagram of a non-limiting example 100 of an environment that is operable to employ cardiac monitoring with sleep and activity correlation as described herein. The illustrated example 100 includes a person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102, such as to generate measurements 108. In some scenarios, for instance, the monitoring device 104 may be provided to record electrical activity of the person 102's heart over an observation period, e.g., lasting some number of seconds or minutes, lasting multiple days, and so on. By way of example, the person 102 may have a magnitude of his or her heart's electrical potential monitored over time to produce one or more electrocardiograms, which may be used to predict any of a variety of events. In one or more implementations, alternatively or in addition, the monitoring device 104 may be provided to record accelerometer data over the observation period, such as to collect movement and activity data for sleep/wake detection, activity level classification, and correlation with cardiac events. In one or more implementations, the accelerometer data may be sampled at a low sampling rate (e.g., in a range between 1-5 Hertz (Hz), such as 1.6 Hz) to enable longer battery life while maintaining accurate detection capabilities for sleep and activity patterns. The monitoring device 104 may output the measurements 108 (e.g., a time sequence of measurements, such as a time sequence of electric potential measurements, acceleration measurements, and/or other types of physical and/or physiological measurements), which may indicate an observation or be used to generate a prediction of one or more events.

In connection with the monitoring device, instructions may be provided to the person 102 that instruct the person 102 how to operate the monitoring device 104 and/or how to behave (e.g., sleep, perform activity) while wearing monitoring device 104, examples of which will be elaborated herein. In one or more implementations, the instructions may be provided as part of a kit, e.g., written instructions. Alternatively or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of the observation period has lapsed (e.g., two days) while wearing the monitoring device 104 and/or based on patterns in the aspects of the person 102 being measured.

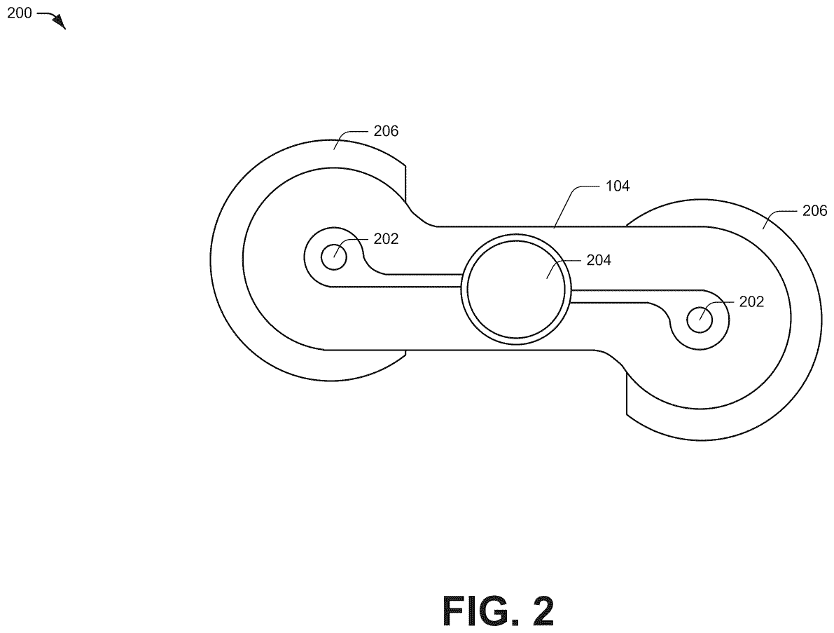

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person 102), an accelerometer, and a pulse oximeter (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner to monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively or additionally, the monitoring device 104 may have a similar form factor as for clinical settings but may have different functionality, such as functionality that prevents a wearer from viewing the measurements 108.

As used herein, the term “continuous” used in connection with monitoring any signals associated with the person 102 (e.g., acceleration data and/or electrical activity of the person 102's heart) may refer to an ability of a device to produce measurements substantially continuously, such that the device may be configured to produce the measurements 108 at intervals of time (e.g., every hour, every 30 minutes, every 5 minutes, every minute, every 30 seconds, every second, every half second, and so forth), responsive to an event (e.g., an electrical signal reaching an inflection point such as a peak or a valley), and so forth. The functionality of the monitoring device 104 to produce the measurements 108 along with other measurements and/or to record any of a variety of signals may vary without departing from the spirit or scope of the described techniques.

In one or more implementations, the monitoring device 104 may be configured to offload measurements 108 during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of sleep patterns, activity levels, arrhythmias, and/or cardiac wellness. By way of example, the monitoring device 104 may perform light processing of the measurements 108 during wear, such as by averaging or encoding at least a portion of the measurements 108, to reduce the amount of data stored and/or transmitted while preserving information for post-wear analysis. In some such implementations, more comprehensive data processing may occur after the wear period to conserve battery power and reduce memory storage usage.

In one or more implementations, the monitoring device 104 may also implement other power management strategies where data from one type of sensor is used to selectively trigger measurement capture by other sensors. As an illustrative example, additionally or alternatively, accelerometer data indicating sleep/wake transitions or specific activity levels may trigger enhanced cardiac monitoring data during periods of interest, enabling analysis of whether cardiac arrhythmias such as atrial fibrillation, heart block, or ventricular arrhythmias correlate with sleep stages, activity levels, and/or exercise intensity. This selective triggering approach, for instance, may enable ambulatory cardiac stress testing by correlating exercise-related activity patterns with concurrent cardiac rhythm changes. In one or more implementations, the monitoring device 104 may implement sleep/activity triggering of analysis and/or data acquisition, where detected sleep stages may trigger different cardiac monitoring modes, such as reduced sampling rates during deep sleep periods or enhanced arrhythmia detection during rapid eye movement (REM) sleep phases. Similarly, detected activity levels may trigger adaptive data acquisition strategies, such as increased cardiac monitoring resolution during exercise periods or modified analysis algorithms during sedentary periods. This sleep/activity triggering approach may optimize battery usage while ensuring comprehensive data capture during physiologically relevant periods. Additional details of the sleep/activity triggering approach will be described below.

To the extent that the monitoring device 104 may be configured to store the measurements 108 for an entirety of the observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the device's storage.

Accordingly, the monitoring device 104 may be configured with one or more ports to enable wired transmission of the measurements 108 to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternatively or additionally be configured to offload the measurements 108 over one or more wireless connections.

Once the monitoring device 104 produces the measurements 108, the measurements 108 are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from the device's local storage to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured to generate the one or more predictions 110. For example, the measurements 108 may be processed by a smartphone associated with the user, a smartphone or other dedicated device associated with the monitoring device 104, and/or one or more server computers at a data center or other location that can be utilized by an entity associated with the monitoring device 104, to name just a few. In other words, those other devices may implement at least a portion of the analysis platform 106 and/or the prediction system 114.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104, or the monitoring device 104 may be inserted into an apparatus having a receptacle that interfaces with corresponding contacts of the monitoring device 104. The measurements 108 may be obtained from a storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternatively or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth (e.g., Bluetooth Low Energy links), near-field communication (NFC), Long Term Evolution (LTE) standards such as 5G, and so forth. Monitoring devices 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of the observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, etc. As noted above, examples of such additional measurements include but are not limited to oxygen saturation (SpO2) measurements. In one or more implementations, the combination of accelerometer data and cardiac measurements may enable enhanced arrhythmia correlation capabilities, including the ability to correlate cardiac arrhythmias such as atrial fibrillation, ventricular arrhythmias, or bradycardia episodes with concurrent sleep stages and/or activity levels. In one or more implementations, the analysis performed by the analysis platform 106 may include correlation analysis between sleep/wake patterns and cardiac arrhythmias to identify patterns where specific sleep stages may predispose users to arrhythmias or where activity levels may trigger cardiac irregularities. Additionally or alternatively, the analysis platform 106 may correlate exercise intensity to cardiac events, enabling identification of which activity intensities may precipitate cardiac arrhythmias or other physiological responses, supporting ambulatory cardiac stress testing capabilities. The sleep and activity context may provide insights not achievable with separate analyses, such as determining optimal exercise intensities for particular users or patient populations while monitoring for exercise-related arrhythmias.

In one or more implementations, the analysis platform 106 may be implemented in whole or in part at the monitoring device 104. Alternatively or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform includes a storage device 112. In accordance with the described techniques, the storage device 112 may be configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In one or more implementations, the storage device 112 may also store sleep/wake detection parameters, activity thresholds, arrhythmia classification algorithms, algorithms for sleep and activity correlation, historical sleep and activity patterns, baseline accelerometer data for comparison purposes, body angle calculation parameters, and multi-modal data fusion algorithms that determine how to combine accelerometer data with other physiological measurements such as ECG or SpO2 data for enhanced prediction accuracy. One or more algorithms may be or may include machine learning algorithms. By way of example, at least one machine learning algorithm may be trained to recognize temporal relationships between sleep stages and/or activity levels and cardiac events. In one or more implementations, the storage device 112 may also maintain arrhythmia classification models, sleep-arrhythmia correlation databases, cardiac event timing relative to sleep and activity patterns, exercise-related arrhythmia detection algorithms, and/or algorithms for identifying causal relationships between specific sleep stages and/or activity intensities and arrhythmia types. The storage device 112 may further store sleep/activity triggering parameters that define how different sleep stages and activity levels should trigger specific analysis modes, data acquisition strategies, and processing algorithms, enabling adaptive monitoring based on physiological context.

In the illustrated example 100, the analysis platform 106 also includes the prediction system 114. The prediction system 114 represents functionality to process the measurements 108 to generate the one or more predictions 110. Alternatively or in addition, the prediction system 114 may output one or more time sequences indicating an observation or prediction of one or more events over time. It is also to be appreciated that the prediction system 114 may output different combinations of multiple predictions in variations.

In at least one implementation, the prediction system 114 uses machine learning to generate at least a portion of the one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively or additionally, the prediction system 114 may include logic (a machine learning model and/or other types of logic) to preprocess the measurements 108, such as to extract various cardiovascular, movement, and/or other features from the sequences of measurements. The one or more predictions 110 may be the output of the prediction system 114, for example.

In various examples, the prediction system 114 may be representative of and/or may include an activity detection system 116 and a cardiac monitoring system 118, and the one or more predictions 110 may include and/or may be representative of a sleep stage classification 120, an activity level classification 122, a cardiac rhythm classification 124, an arrhythmia correlation 126, and/or a cardiac wellness prediction 128. For instance, as further described in more detail below, the activity detection system 116 may be operable to implement accelerometer-based techniques to generate the sleep stage classification 120 and the activity level classification 122. The activity detection system 116 may receive accelerometer data and detect peaks above a threshold to identify steps, analyze movement patterns, analyze body angle, and/or analyze activity intensity to classify sleep stages and wake states as part of the sleep stage classification 120, and classify activity levels within predetermined epochs as part of the activity level classification 122. The activity level classification 122, for instance, may categorize activity levels as sedentary, light, moderate, or vigorous based on step counts within the predetermined epochs and/or accelerometer features and movement patterns extracted from the accelerometer data. By way of example, the activity detection system 116 may incorporate time-domain and frequency-domain features of the accelerometer data for enhanced activity classification.

The cardiac monitoring system 118 may be operable to analyze cardiac data patterns and correlate them with sleep and activity data to generate the cardiac rhythm classification 124 and the arrhythmia correlation 126, which may indicate the timing and context of cardiac events, including their correlation with sleep stages and/or activity levels. In one or more implementations, the cardiac monitoring system 118 may leverage cardiac data of the measurements 108 (e.g., ECG waveform data) to identify rhythm types and correlate them with concurrent accelerometer data of the measurements 108, providing context for arrhythmia diagnosis and patient care. The cardiac monitoring system 118 may employ one or more algorithms (e.g., machine learning algorithms and/or conventionally programmed algorithms) to analyze the cardiac data, classifying cardiac rhythms and identifying arrhythmias by considering factors such as heart rate variability, rhythm patterns, and morphological features. The cardiac monitoring system 118 may further employ one or more algorithms (e.g., machine learning algorithms and/or conventionally programmed algorithms) to analyze sleep and activity data of the measurements 108 (e.g., accelerometer data) to provide contextual information for cardiac events, resulting in the arrhythmia correlation 126. Alternatively or in addition, the prediction system 114 may combine analyses performed by the activity detection system 116 and the cardiac monitoring system 118 to output the arrhythmia correlation 126 and/or the cardiac wellness prediction 128.

In one or more implementations, the prediction system 114 may implement sleep/activity triggering of analysis and/or data acquisition, where different sleep stages and activity levels trigger adaptive monitoring strategies. By way of example, when activity is detected by the activity detection system 116, the monitoring device 104 may turn on additional channels of data acquisition such as a gyroscope, which are more costly in terms of battery consumption but may provide additional insight into the type or intensity of the activity performed. When sleep is detected by the activity detection system 116, more battery-intensive data acquisition such as LED-based photoplethysmography (PPG) for SpO2, which may be used for sleep apnea screening and diagnosis, may be triggered. The LED-based PPG may turn off when the person 102 is no longer asleep. As another example, the cardiac monitoring system 118 may further utilize sleep and activity detection to enable selection of baseline events or scheduled events that provide relatively clean data measurements (e.g., ECG strips) for analysis. Similarly, detect noise-generating activities may enable the prediction system 114 to label noisy ECG data for exclusion from cardiac rhythm analysis, thereby improving the accuracy of the cardiac rhythm classification 124 and arrhythmia correlation 126. Detecting sleep and activity during wear for the monitoring device 104 may further enable reporting of additional context information during the observation period, such as nocturnal heart rates, nocturnal heart rate variability, and other wellness information.

In one or more implementations, the cardiac monitoring system 118 may be configured to identify specific arrhythmia types and correlate them with concurrent sleep or activity states, which may be output as the arrhythmia correlation 126. By way of example, the arrhythmia correlation 126 may indicate bradycardia episodes that occur during sleep, atrial fibrillation episodes that correlate with specific activity levels, and/or ventricular arrhythmias that may be triggered by exercise intensity or sleep transitions, just to name a few non-limiting examples. The cardiac monitoring system 118 may also analyze whether sleep stage transitions themselves trigger cardiac arrhythmias, whether specific activity intensities precipitate cardiac irregularities, and/or correlations between sleep quality and cardiac rhythm stability. The cardiac monitoring system 118 may further correlate exercise intensity with cardiac arrhythmias to enable ambulatory cardiac stress testing, providing insights into exercise-related cardiac risk patterns that are not achievable through separate analysis of cardiac monitoring and activity tracking. By integrating the activity detection system 116 with the cardiac monitoring system 118, the prediction system 114 may uncover relationships between cardiac events and physiological states, offering healthcare providers a comprehensive tool for assessing patient cardiac health in the context of daily activities and sleep patterns.

By way of example, the cardiac wellness prediction 128 may provide comprehensive physiological insights that combine sleep and activity analysis with cardiac context, enabling the detection of relationships between sleep patterns, activity levels, cardiac wellness metrics, and overall health status. The cardiac wellness prediction 128 may include correlations between sleep quality and cardiac health indicators, relationships between daily activity levels and cardiac function, and patterns suggesting cardiovascular fitness, exercise tolerance, or potential health risks. For instance, the cardiac wellness prediction 128 may indicate relationships between sleep duration and heart rate variability, correlations between activity intensity and cardiac arrhythmia frequency, or associations between sleep/wake patterns and overall cardiovascular wellness. The cardiac wellness prediction 128 may also provide insights into how different sleep stages and activity levels affect cardiac function over time, including information about exercise capacity, recovery patterns, and the impact of sleep quality on heart health. By integrating accelerometer-derived sleep and activity data with concurrent cardiac measurements, the cardiac wellness prediction 128 may identify optimal sleep and activity patterns for individual users, indicate early signs of cardiovascular decline, and/or provide personalized recommendations for maintaining or improving overall cardiac health.

Alternatively or in addition, the cardiac wellness prediction 128 may include ambulatory cardiac stress test results, where the activity detection system 116 detects exercise periods from accelerometer data and the cardiac monitoring system 118 analyzes concurrent cardiac responses from electrical potential data to identify exercise-induced arrhythmias and cardiac recovery patterns that may indicate underlying coronary heart disease or other cardiovascular conditions. The cardiac wellness prediction 128 may include evaluations of chronotropic incompetence by comparing expected and actual heart rate responses during detected activity events, providing insights into cardiac function during physical exertion. The cardiac wellness prediction 128 may further generate exercise prescription recommendations and stress test insights based on the observed relationships between specific activity intensities and cardiac wellness metrics, enabling personalized exercise guidance that promotes cardiovascular benefits while minimizing arrhythmia risk for individual users or patient populations. This multi-modal approach may provide clinical benefits by supporting preventive cardiac care strategies, enabling early intervention when certain patterns are detected in the combined sleep, activity, and cardiac data, and facilitating ambulatory cardiac stress testing through correlation of exercise intensity with cardiac response patterns without an in-office visit.

Further illustrated in the example 100 is an accessory device 130 and a healthcare provider 132. The accessory device 130, for instance, may include one or more devices associated with the person 102 and/or the monitoring device 104, such as those described above. For instance, the accessory device 130 may include a display device (e.g., a smartphone and/or a personal display device) to display the one or more predictions 110 and/or to control functionality of the monitoring device 104. The accessory device 130 may also display a notification 134 and/or instructions 136, as will be further elaborated below.

The healthcare provider 132, for instance, may be representative of one or more additional processing devices associated with an authorized medical system, e.g., practitioner devices, electronic health record systems, diagnostic imaging equipment, laboratory information systems, telemedicine platforms, clinical decision support tools, and so forth. In various implementations, the notification 134 may be generated based on the one or more predictions 110, such as alerts for detected arrhythmias, changes in sleep patterns, changes in activity levels, changes in the cardiac wellness prediction 128, exercise-related cardiac events, and/or other detected events, and may be displayed by the accessory device 130 to communicate information to the person 102, healthcare providers, caregivers, and/or emergency contacts. As a non-limiting example, the prediction system 114 may generate reports and/or alerts based on the arrhythmia correlation 126, which may lead to more targeted cardiac interventions and improved cardiac care strategies.

In connection with the monitoring device 104, the instructions 136 may be provided to the person 102 to instruct the person 102 how to operate the monitoring device 104 and/or tasks to perform (e.g., sleep, rest, or perform an activity) while wearing monitoring device 104. The instructions 136 may help ensure sufficient data is acquired for cardiac stress testing by specifying specific durations and/or intensities of physical activity and/or recovery (e.g., recovery in the supine position). In one or more implementations, clinical workflows may be modified to collect exercise data while the person 102 is wearing the monitoring device 104 rather than (or in addition to) having the person 102 perform a separate in-office cardiac stress test with 12-lead ECG cardiac monitoring. By way of example, the instructions 136 may provide specific exercise regimens and recovery during wear of the monitoring device 104 in order to collect specific data for the cardiac stress test.

In various examples, one or more operations of the analysis platform 106, the prediction system 114, the activity detection system 116, and/or the cardiac monitoring system 118 are performable by one or more of the monitoring device 104, the accessory device 130, the devices and systems of the healthcare provider 132, and/or one or more additional devices not shown.

FIG. 2 depicts a non-limiting example 200 of a monitoring device. The illustrated example 200 depicts the monitoring device 104.

In accordance with the described techniques, the monitoring device 104 includes one or more sensors 202, examples of which include but are not limited to one or more pairs of electrodes, an accelerometer, a pulse oximeter, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the person 102's heart, e.g., to produce an electrocardiogram (ECG and/or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as the person 102's heart's electrical activity. In one or more implementations, the processor further generates one or more communicable packages of data that include one or more of the measurements 108 and/or other measurements, such oxygen saturation (SpO2) measurements. Alternatively or additionally, the processor produces and/or causes storage of other data, which may be used for predicting classifications of physiological conditions, e.g., arrhythmias, activity levels, and the like.

In implementations where the monitoring device 104 is configured for wireless transmission, the transmitter 204 may transmit the measurements 108 wirelessly as a stream of data to a computing device. In one or more implementations, for instance, the monitoring device 104 is configured to transfer (e.g., transmit and/or receive) information (e.g., electrical potential measurements) via a Bluetooth Low Energy (BLE) connection. Alternatively or additionally, the monitoring device 104 may buffer the measurements 108 (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

Cardiac Monitoring System with Sleep and Activity Correlation

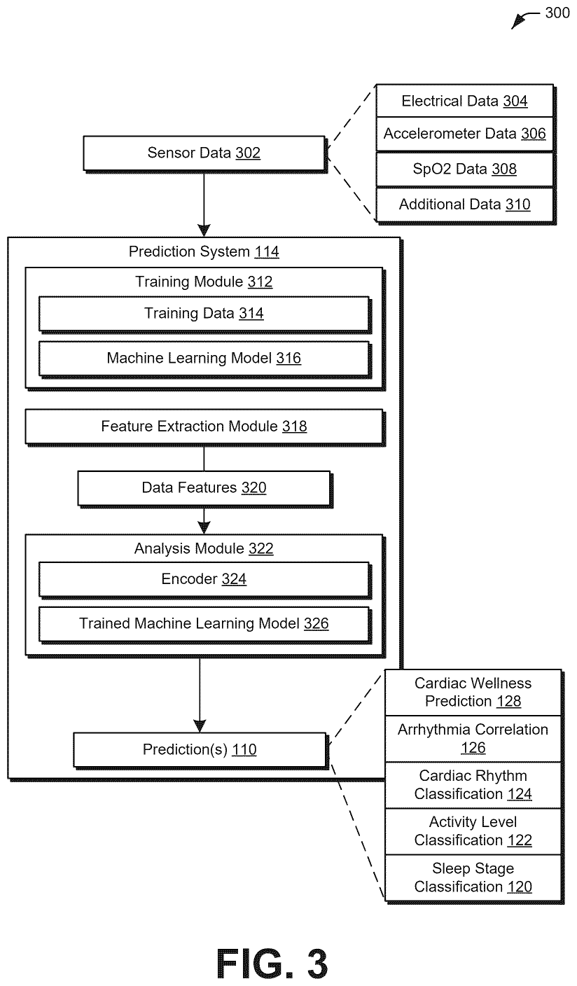

FIG. 3 depicts a non-limiting system in an example implementation 300 of cardiac monitoring with sleep and activity correlation showing operation of the prediction system 114 of FIG. 1 in more detail.

To begin in this example, the prediction system 114 receives sensor data 302, which may include electrical data 304 (e.g., electrical potential measurements and/or ECG data), accelerometer data 306, SpO2 data 308 (e.g., oxygen saturation data), and/or various additional data 310. In various examples, the sensor data 302 is collected by one or more devices and/or sensors, such as the wearable monitoring device 104. The sensor data 302 can include time-sequenced instances of data, such as continuous data, data collected at predetermined intervals (e.g., per half-second interval, per minute interval, per five minute interval, etc.) for the length of an observation period, e.g., a single day, multiple days during a week, for a month, and so forth. The sensor data 302 may be the measurements 108 introduced with respect to FIG. 1, for example. In one or more implementations, the accelerometer data 306 is collected at a sampling rate that is less than 5 Hz (e.g., a 1.6 or 1.56 Hz sampling rate). Generally, the sensor data 302 is processed by the prediction system 114 to generate the one or more predictions 110, which may include one or more of the sleep stage classification 120, the activity level classification 122, the cardiac rhythm classification 124, the arrhythmia correlation 126, and the cardiac wellness prediction 128, in accordance with the techniques described in more detail below.

For instance, the prediction system 114 includes a training module 312 that is operable to train a machine learning model 316 using training data 314 to perform one or more sleep stage classification, activity classification, and/or cardiac monitoring tasks. The sleep stage classification, activity classification, and cardiac monitoring tasks, for instance, involve generation of the one or more predictions 110, such as the cardiac rhythm classification 124 based on patterns in the electrical data 304, correlating sleep stage and activity patterns with cardiac data from the electrical data 304 for the arrhythmia correlation 126 and the cardiac wellness prediction 128, and monitoring body position and sleep stages based on the accelerometer data 306 for the sleep stage classification 120. These tasks may include classifying sleep stages (such as light sleep, deep sleep, REM sleep, and wake states), classifying activity levels (such as sedentary, light, moderate, or vigorous), detecting cardiac arrhythmias, and correlating the arrhythmias with sleep stage and activity data (e.g., based on the accelerometer data 306). By way of example, identifying cardiac arrhythmias from the electrical data 304 that may correlate with specific sleep stages or activity levels may enable the analysis of relationships between sleep stage patterns, exercise intensity, and arrhythmia occurrences. Accordingly, the machine learning model 316 is trained to correlate patterns in the sensor data 302, such as various electrical potential measurements of the electrical data 304 and/or accelerometer measurements and body angle calculations of the accelerometer data 306, to the one or more predictions 110. It is to be appreciated that more than one machine learning model 316 may be separately trained, such as separate machine learning models 316 for the different one or more predictions 110.

The previous examples describe various instances of artificial intelligence (“AI”) models and/or machine learning models. In one or more examples, an AI model, e.g., a machine learning model, refers to a computer representation that is tunable (e.g., through training and retraining) based on inputs without being actively programmed by a user to approximate unknown functions, automatically and without user intervention. For instance, the term “machine learning model” includes a model that utilizes algorithms to learn from, and make predictions on, known data by analyzing training data to learn and relearn to generate outputs that reflect patterns and attributes of the training data. In the context of cardiac monitoring with sleep stage and activity correlation, machine learning models are implementable (e.g., by one or more processing devices of the prediction system 114) to analyze accelerometer data patterns and correlate them with cardiac data to identify sleep stages, activity levels, detect cardiac arrhythmias, and generate comprehensive physiological insights.

The training module 312 is further operable to initialize various parameters of the machine learning model 316, which are usable by the machine learning model 316 as internal variables to represent and process information during training. These parameters are further usable to represent inferences gained through training. In one or more implementations, the training data 314 are separated into batches to improve processing and optimization efficiency of the parameters of the machine learning model 316 during training, which is particularly beneficial for model accuracy when processing large volumes of accelerometer time-series data collected at low sampling rates and correlating sleep stage and activity patterns with concurrent cardiac measurements.

In the present example, the training data 314 may include historical electrical potential measurements (e.g., such as ECG) and accelerometer measurements data from a population of users along with corresponding historical outcome data, such as arrhythmia classifications, activity classifications, sleep stage classifications, and/or overall cardiac wellness classifications. These data may be collected from clinical studies, sleep monitoring studies, cardiac monitoring studies, clinical cardiac stress tests, or other sources where the electrical data 304 and the accelerometer data 306 are recorded simultaneously. In some cases, the training data 314 may also include additional physiological measurements such as oxygen saturation levels (e.g., the SpO2 data 308), and/or additional relevant biomarkers. The training data 314 may be labeled with various sleep stage, activity, and cardiac-related information, such as the presence or absence of sleep periods, types of activities (e.g., walking, running, sleeping), activity levels, sleep stages (such as light sleep, deep sleep, REM sleep, and wake states), cardiac arrhythmias, body positions, and so forth.

The training data 314 may also be labeled with various heart rhythm-related information. That is, in order to train the machine learning model for cardiac monitoring with sleep stage and activity correlation, the training data 314 may provide examples of “what is to be learned” by the machine learning model, e.g., as a basis to learn patterns from the data. For cardiac monitoring applications, the training data 314 may include labeled datasets of accelerometer measurements and cardiac data from users with known abnormal sleep stage patterns, activity levels, and cardiac conditions, as well as measurements from individuals with normal sleep stage patterns, activity levels, and no cardiac abnormalities. The training module 312, for instance, may collect and preprocess the training data 314 that includes input features (e.g., accelerometer waveforms, body angle calculations, sleep stage patterns, activity patterns, ECG data, heart rate patterns) and corresponding target labels (e.g., “deep sleep stage,” “vigorous activity,” “atrial fibrillation detected,” “arrhythmia correlated with exercise,” or specific sleep stage, activity, and cardiac classifications). The training data 314 may further be labeled with physiological features such as normal sinus rhythm, atrial fibrillation episodes, ventricular arrhythmias, bradycardia events, heart block occurrences, premature ventricular contractions, supraventricular tachycardia, and/or other cardiac rhythm abnormalities that may be temporally associated with sleep stages or activity level changes.

The training data 314 may be received as an input by the machine learning model 316 and used as a basis for generating predictions based on a current state of parameters of layers and corresponding nodes of the model, a result of which is output as output data. A machine learning model, for instance, may be configurable using a plurality of layers having, respectively, a plurality of nodes. The plurality of layers is configurable to include an input layer, an output layer, and one or more hidden layers. In the context of cardiac monitoring with sleep stage and activity correlation, the input layer may receive the sensor data 302 or features thereof, including magnitude calculations, body angle measurements, step detection peaks, movement patterns, activity level indicators, and cardiac features. The hidden layers, for instance, process these inputs through weighted connections to identify complex patterns indicative of sleep stages, activity levels, cardiac arrhythmias, and correlations between sleep stages/activity and cardiac status, e.g., patterns that are not detectable using conventional analysis modalities that rely solely on basic cardiac monitoring or activity tracking. The output layer may produce the one or more predictions 110, including the sleep stage classification 120, the activity level classification 122, the cardiac rhythm classification 124, the arrhythmia correlation 126, and/or the cardiac wellness prediction 128. Calculations are performed by the nodes within the layers via hidden states through a system of weighted connections that are “learned” during training of the machine learning model 316 to implement a variety of sleep stage detection, activity detection, and cardiac monitoring tasks.

In some implementations, different training schemes and/or model architectures are employed based on what the one or more predictions 110 are to include. For instance, a composition and structure of the training data 314 may vary depending on the specific type of prediction to be generated. In an example in which the one or more predictions 110 are to indicate a binary classification of sleep/wake state (e.g., whether a user is asleep or awake), the training data 314 may be labeled with yes/no indicators. In an additional or alternative example in which the one or more predictions 110 are to include granular predictions such as specific sleep stages (light sleep, deep sleep, REM sleep), activity levels, and/or arrhythmia type, the training data 314 include detailed annotations that pertain to the granular predictions. In an example in which the one or more predictions 110 are to indicate correlations between sleep stage/activity patterns from the accelerometer data 306 and cardiac wellness metrics from the electrical data 304, the training data 314 may include wellness insights associated with various sleep stages, activity levels, and cardiac measurements. In an additional or alternative example in which the one or more predictions 110 are to include comprehensive analyses such as ambulatory cardiac stress testing using exercise detection and cardiac response monitoring, sleep stage-arrhythmia correlations, and/or exercise-related arrhythmia detection with physiological context from the electrical data 304, the training data 314 may include multi-modal input data in order to capture the complex relationships between sleep stages, activity, cardiac status, and physiological outcomes.

In some examples, the training data 314 are structured to support multi-task learning, where the machine learning model 316 can simultaneously predict multiple aspects of sleep stage classification, activity classification, and/or cardiac monitoring, such as sleep stage and activity level in combination, as well as sleep stage-cardiac correlations and arrhythmia detection with physiological context, such as cardiac wellness insights from sleep stage-heart rate relationships and/or exercise-related arrhythmia patterns from activity-cardiac data combinations. In additional or alternative examples, the training module 312 trains the machine learning model 316 on a per task basis, such as to implement a first round of training to train the machine learning model 316 to perform sleep stage-cardiac correlation analysis and a second round of training to train the machine learning model 316 to perform exercise-related arrhythmia detection with activity contextualization. In this way, the techniques described herein support targeted training of the machine learning model 316 for particular tasks, which improves model performance and efficiency to perform discrete aspects of sleep stage detection, activity classification, and cardiac monitoring as well as complex multi-modal physiological insights that combine the accelerometer data 306 with the electrical data 304 rather than basic sleep stage or cardiac monitoring alone.

In one or more implementations, the training module 312 trains the machine learning model 316 using an iterative process of adjusting weights and learning parameters to minimize a loss function. For example, the training module 312 may use backpropagation and/or gradient descent algorithms to update parameters of the model based on a difference between predicted and actual sleep/wake states, activity classifications, and cardiac arrhythmia classifications in the training data 314. A learning rate, batch size, and/or number of epochs may be tuned to optimize the performance of the machine learning model 316.

Training of the machine learning model 316 can include calculation of a loss function to quantify a loss associated with operations performed by nodes of the machine learning model 316. The loss function is configurable in various ways to control operation and/or functionality of the machine learning model 316. For instance, the loss function may be designed to prioritize accuracy in detection of cardiac arrhythmias while minimizing false positives, and to optimize correlation accuracy between sleep stage/activity patterns and cardiac wellness metrics. Calculation of the loss function, for instance, includes comparing a difference between predictions specified in the output data (e.g., predicted sleep stages, activity levels, cardiac arrhythmias, or sleep stage-cardiac correlations) with target labels specified by the training data 314 (e.g., clinically confirmed sleep stage classifications, activity classifications, and documented cardiac arrhythmias). The loss function is configurable in a variety of ways, examples of which include a quadratic loss function as part of a least squares technique for continuous cardiac parameters, cross-entropy loss for classification tasks such as sleep stage categorization, custom loss functions that incorporate clinical risk factors specific to arrhythmia detection and sleep stage-cardiac correlations, and so forth.

Furthermore, a variety of architectures/types of the machine learning model 316 are considered. In one or more implementations, the machine learning model 316 may include a neural network, such as a convolutional neural network (CNN), recurrent neural network (RNN), or a combination thereof. In some instances, the machine learning model 316 incorporates one or more U-Net and/or ResNet architectures, features, or components. The model may also be implemented as an ensemble of different algorithms that combines one or more decision trees, random forests, and/or gradient boosting machines with neural network approaches. By way of example, CNNs may be used for analyzing accelerometer waveform patterns and detecting sleep/wake signatures. As another example, long short-term memory (LSTM) neural networks may be used to analyze temporal sleep and activity patterns and correlate activity sequences with cardiac events. In still other examples, generative adversarial networks (GANs), decision trees (e.g., for sleep/wake classification and cardiac arrhythmia assessment), support vector machines, linear regression, logistic regression for binary sleep/wake detection, Bayesian networks, random forest learning for feature importance in accelerometer and cardiac data correlation, dimensionality reduction algorithms, boosting algorithms, deep learning neural networks, and so forth may be employed. It is to be appreciated that the above examples are given by way of illustration, and other configurations may be used without departing from the spirit or scope of the described techniques.

In some examples, techniques such as dropout or regularization may be employed by the training module 312, such as to prevent overfitting. The training process may continue until the model achieves a desired level of accuracy on a validation dataset and/or until a predetermined number of iterations have been completed. This approach allows the machine learning model 316 to learn complex patterns in the sensor data 302 that are indicative of relationships between sleep/activity patterns from the accelerometer data 306 and cardiac wellness metrics from the electrical data 304, correlations between sleep stage changes and heart rate responses during sleep transitions, temporal associations between exercise intensity and cardiac arrhythmias, and multi-modal insights that combine sleep, activity, and cardiac data to identify subtle features and develop physiological understanding that is not possible using conventional analysis methods. By way of example, it may be impractical or impossible to manually analyze the sensor data 302 to arrive at such insights. This is by way of example and not limitation, and a variety of suitable training techniques are considered.

Multiple attention heads, for instance, may allow the machine learning model 316 to allocate resources to focus on different aspects of the input data to make distinct predictions. Continuing with the above example in which the predictions 110 indicate sleep-cardiac correlations, each analysis head may be trained to detect specific relationships between sleep/activity patterns from the accelerometer data 306 and physiological responses from the electrical data 304, which enables the prediction system 114 to provide comprehensive multi-modal analysis of the sensor data 302 for cardiac wellness insights and ambulatory cardiac stress testing. In this way, the techniques described herein support adaptability of the prediction system 114 to efficiently provide focused sleep-cardiac correlation information and arrhythmia detection with physiological context.

Once the machine learning model 316 is trained, the sensor data 302 is processed by a feature extraction module 318, which generates data features 320. For instance, the feature extraction module 318 preprocesses the sensor data 302 to generate usable (e.g., processable) inputs for the trained machine learning model 326. The feature extraction module 318 may generate at least a portion of the data features 320 based on various properties of the accelerometer signal present in the accelerometer data 306, such as magnitude calculations, body angle measurements, sleep/wake indicators, movement patterns, and/or activity level indicators. These extracted features can include time-domain, frequency-domain, and statistical measures that capture relevant information about sleep stages, movement, and body position. It is to be appreciated that the training data 314 may include accelerometer data obtained at the same sampling frequency as the accelerometer data 306, at least in some implementations.

The feature extraction module 318 may be further operable to perform body angle calculations as a part of outputting the data features 320. In some implementations, body angle techniques may be used to extract positional information from the accelerometer data 306 to determine body position changes and sleep/wake states. The feature extraction module 318 may analyze variations in accelerometer orientation, such as changes in gravitational vector components, which can be correlated to and/or influenced by body position, sleep stages, and movement activity. These position-related properties of the accelerometer data 306 may be used to derive body angle measurements, sleep/wake patterns, and activity patterns. The body angle-derived features may also be combined with other data features 320 to provide a comprehensive set of inputs for the trained machine learning model 326. In this way, the prediction system 114 is able to capture both movement and positional information from a single accelerometer signal, which improves detection of correlation between body position, sleep stages, and activity events and is not possible using conventional modalities.

In one or more implementations, the feature extraction module 318 may also extract cardiac features from the electrical data 304, such as heart rate variability, QRS complex morphology, R-R interval patterns, and arrhythmia signatures that may be correlated with sleep stages or activity level changes.

The feature extraction module 318 may further implement a variety of additional techniques such as wavelet decomposition, principal component analysis, peak detection, statistical analysis, and/or other signal processing methods to isolate and quantify relevant aspects of the electrical data 304 and/or the accelerometer data 306. For example, step detection algorithms may be applied to identify peaks above predetermined thresholds, while magnitude calculations may provide information about overall activity levels. The feature extraction module 318 may incorporate both time-domain features (such as mean, variance, and peak characteristics) and frequency-domain features (such as spectral energy and dominant frequencies) for enhanced sleep/wake and activity classification. The feature extraction module 318 may further optimize and/or refine the data features 320, such as based on a discriminative ability of the data features 320 to detect particular sleep stages, activity levels, or cardiac events.

Once extracted, an analysis module 322 configures the data features 320 for input to a trained machine learning model 326 (e.g., the machine learning model 316 once output by the training module 312). In one or more implementation, the analysis module 322 uses an encoder 324. The encoder 324 is configurable to process and compress input data into a compact representation and can include one or more of a convolutional encoder, recurrent encoder, transformer encoder, one or more autoencoder variants, and so forth. The encoder 324, for instance, generates compressed representations from the data features 320 that can be efficiently processed by the trained machine learning model 326. In an example, the encoder 324 reduces a dimensionality of the data features 320 while preserving relevant information, creating a compact representation that serves as a suitable input to the trained machine learning model 326.

The analysis module 322 generates the one or more predictions 110 for output by processing the encoded data features 320 using the trained machine learning model 326. The predictions 110 may include a variety of information. By way of example, the sleep stage classification 120 may indicate sleep stages, the activity level classification 122 may indicate different levels of physical activity during wake states, the cardiac rhythm classification 124 may indicate detection of cardiac arrhythmias, the arrhythmia correlation 126 may indicate correlations between cardiac events and sleep/activity states, and the cardiac wellness prediction 128 may provide comprehensive physiological insights that combine sleep and activity analysis with cardiac context and/or additional predictions that provide supplementary analysis results. In one or more examples, the one or more predictions 110 may include a confidence interval, e.g., a confidence in the associated value or condition.

In at least one implementation, the sleep stage classification 120 may include classification of sleep and wake states by analyzing patterns in the accelerometer data 306 to identify sleep and wake periods and may provide accurate sleep/wake detection while operating at lower sampling frequencies compared to conventional systems. The activity level classification 122 may distinguish between levels of activity including but not limited to sedentary, light activity, moderate activity, vigorous activity, or combinations thereof. The activity level classification 122 may classify activity intensity based on movement patterns and/or step counts within predetermined epochs, incorporating extracted features from the accelerometer data 306 such as time-domain and frequency-domain characteristics. As a non-limiting example, movement equivalent to walking speed of 2 miles per hour (mph) or greater may indicate light activity, whereas speeds of less than 2 mph may indicate inactivity/rest. The cardiac rhythm classification 124, for instance, may indicate whether a user associated with the sensor data 302 has experienced normal cardiac rhythms, specific types of arrhythmias (e.g., atrial fibrillation, bradycardia, ventricular arrhythmias), and the timing of cardiac events, just to name a few.

The arrhythmia correlation 126 may include an indication of a correlation between a cardiac arrhythmia and an additional physiological event, such as a sleep stage, an activity event, and so forth, such as when the trained machine learning model 326 is operable to identify and analyze relationships between arrhythmia occurrences and various sleep stages or physical activity levels. For example, the arrhythmia correlation 126 may indicate that atrial fibrillation events are more likely to occur during specific sleep stages or activity intensities for a particular user. Combining the arrhythmia correlation 126 with the cardiac rhythm classification 124 may indicate temporal associations between specific cardiac arrhythmias and sleep/activity states, such as episodes of atrial fibrillation during REM sleep, bradycardia events during deep sleep periods, or ventricular arrhythmias that may be triggered by exercise intensity. Such insights may offer a comprehensive view of physiological responses and triggers for cardiac events, enabling healthcare providers to better understand whether arrhythmias are related to sleep patterns, activity levels, or other physiological contexts. Moreover, the arrhythmia correlation 126 and cardiac rhythm classification 124 may supply clinicians with detailed information to make informed decisions about cardiac care and lifestyle modifications without relying solely on patient self-reports.

In various examples, the sensor data 302 further include the SpO2 data 308 and/or the various additional data 310. These additional measurements may be input to the machine learning model 316 along with the electrical data 304 and/or the accelerometer data 306 to predict the one or more predictions 110. Accordingly, the techniques described herein support multi-modality predictions that provide insights not capable using conventional techniques that rely solely on the accelerometer data 306 or solely on the electrical data 304.

In some implementations, the SpO2 data 308 may be utilized to enhance the accuracy of and/or validate the sleep, activity, and cardiac predictions. For instance, the analysis module 322 may process the SpO2 data 308 in conjunction with the electrical data 304 and/or the accelerometer data 306 to identify potential sleep periods or activity periods. The trained machine learning model 326 may be configured to detect changes in oxygen saturation levels during sleep periods, which may coincide with sleep apnea events, and correlate these changes with sleep patterns in the accelerometer data 306. Additionally or alternatively, the trained machine learning model 326 may be configured to detect changes in oxygen saturation levels during activity periods and correlate these changes with arrhythmias determined from the electrical data 304. By combining these data sources, the prediction system 114 is operable to distinguish between different types of sleep stages, activity events, and cardiac arrhythmias with enhanced accuracy. This multi-modal approach enables nuanced and accurate sleep, activity, and cardiac predictions, which reduces incidence of false positives and provides additional context for sleep quality, activity levels, and cardiac arrhythmias detected.

The following discussion describes techniques that are implementable utilizing the previously described systems and devices. In portions of the following discussion, reference will be made to FIGS. 1-3.

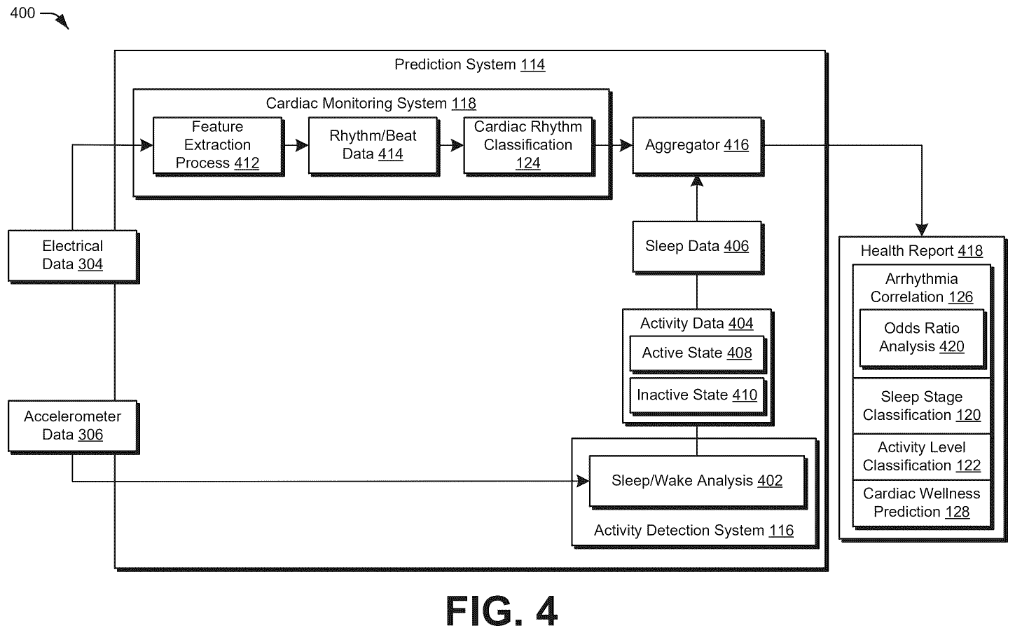

FIG. 4 depicts a monitoring system 400 in a non-limiting example of cardiac monitoring with sleep stage and activity correlation in which the monitoring system 400 generates a comprehensive health report for output. The monitoring system 400 is one implementation of the prediction system 114, for instance, and may utilize the machine learning described with respect to FIG. 3.

To begin in this example, the monitoring system 400 receives the electrical data 304 and the accelerometer data 306 as input. The accelerometer data 306 is first processed at a sleep wake analysis 402 to generate activity data 404 and sleep data 406.

Together, the activity data 404 and the sleep data 406 provide comprehensive information about patient sleep-wake cycles and activity levels throughout the observation period. The activity data 404 is categorized into an active state 408 and an inactive state 410. By way of example, the active state 408 and the inactive state 410 may be label or bins of data in order to distinguish data collected during periods when the person 102 is awake and active versus awake but inactive. Although not shown, the sleep data 406 may be categorized into different sleep stages (e.g., light sleep, deep sleep, REM sleep, non-REM sleep, and so forth).

In parallel, the electrical data 304 is processed through a feature extraction process 412 that generates rhythm/beat data 414. The feature extraction process 412, for instance, extracts relevant cardiac features and patterns from the electrical data 304. The rhythm/beat data 414 may include information about heart rate variability, R-R intervals, QRS complex characteristics, and other temporal and morphological features extracted from the electrical data 304. The cardiac monitoring system 118 may use the rhythm/beat data 414 to generate the cardiac rhythm classification 124, such as by using the trained machine learning model 326. By way of example, the rhythm/beat data 414 may be part of the data features 320.

An aggregator 416 combines outputs from the cardiac monitoring system 118 and the activity detection system 116 to produce a health report 418. By way of example, the aggregator 416 may combine cardiac rhythm analysis with sleep stage and activity state information. The aggregator 416 may synthesize the rhythm/beat data 414, the activity data 404, and the sleep data 406 to generate correlations between cardiac events and patient activity states. The health report 418 generated by the aggregator 416 may include one or multiple analysis outputs for comprehensive patient assessment.

In the illustrated example, the health report 418 includes the arrhythmia correlation 126, the sleep stage classification 120, the activity level classification 122, and the cardiac wellness prediction 128 as integrated outputs. It is to be appreciated, however, that in various scenarios, one or a subset of these outputs may be included in the health report 418. Because the monitoring system 400 processes various types of sensor data, the health report 418 can include a variety of multimodal information, such as correlations between cardiac events and sleep stage patterns, and assessments of cardiac wellness metrics in relation to daily activity patterns and sleep behavior. It is to be appreciated that although the monitoring system 400 is shown processing the electrical data 304 and the accelerometer data 306, other data may also be processed, including the data 308 and the various additional data 310 (e.g., gyroscope data).

An odds ratio analysis 420 may be output as a part of the arrhythmia correlation 126. The odds ratio analysis 420 may highlight trends in arrhythmia occurrence related to sleep-wake cycles and/or activity levels across patient populations. The odds ratio analysis 420 indicates the odds of arrhythmia occurrence during different physiological states by comparing the likelihood of cardiac events during active periods versus inactive periods and/or during sleep periods versus wake periods. The prediction system 114 may process or retrieve data from large patient populations to generate statistically significant insights about arrhythmia patterns, which may provide insights regarding whether specific sleep stages or activity levels increase or decrease the probability of arrhythmia occurrence. The odds ratio analysis 420 may determine, for example, that patients of a specific demographic group or general population are 2.5 times more likely to experience atrial fibrillation during deep sleep stages compared to wake periods, or that ventricular arrhythmias occur 1.8 times more frequently during vigorous activity compared to sedentary periods.

The odds ratio analysis 420 may analyze fourteen rhythm types divided into two groups, where a first group may include second-degree atrioventricular block type 2, complete heart block, atrial fibrillation, bigeminy, trigeminy, ectopic atrial rhythm, idioventricular rhythm, junctional rhythm, pause, supraventricular tachycardia, ventricular tachycardia, and Wenckebach, and a second group may include sinus rhythm and artifacts. The odds ratio analysis 420 may further classify atrial fibrillation devices as high atrial fibrillation for devices with eighty-five percent or greater atrial fibrillation with no sinus rhythm, and low atrial fibrillation for all other atrial fibrillation devices.

In one or more implementations, the odds ratio analysis 420 may use a two-by-two contingency table format with “rhythm” versus “not rhythm” and “sleep” versus “wake” states (and/or particular sleep stages or activity levels), applying the formula:

$$OR = \frac{( A \times D )}{( B \times C )}$$

where the OR is the odds ratio. One or more odds ratio analyses may be generated using the above formula, which may be applied per individual rhythm to determine the odds ratio of that rhythm. In a first example, a time in rhythm analysis is performed, where A represents the time (e.g., in seconds) spent in a specific rhythm being assessed during sleep, B represents the time spent in all rhythms during sleep minus A, C represents the time spent in the specific rhythm during wake, and D represents the time spent in all rhythms during wake minus C. In a second example, an onset of episodes in rhythm analysis is performed, where A represents episodes of the specific rhythm with sleep onset (e.g., the specific rhythm begins during a detected sleep period), B represents episodes of all first group rhythms with sleep onset minus A, C represents episodes of the specific rhythm with wake onset (e.g., the specific rhythm begins during a detected wake period), and D represents episodes of all first group rhythms with wake onset minus C. For each rhythm type, the odds ratio analysis 420 may select data (e.g., analyzed electrical data 304 corresponding to a particular cardiac rhythm classification 124) containing that specific rhythm.

Overall, in at least one implementation, the odds ratio analysis 420 may indicate the proportion of specific rhythms that occur in sleep versus wake, where higher values correspond to higher odds that the rhythm occurs during sleep. The odds ratio analysis 420 may provide clinical insights regarding how the person 102 compares to the general population or another user population separated by age, sex, ethnicity, and/or another demographic indicator. Accordingly, the health report 418 may provide population-level insights as well as or as an alternative to insights regarding the person 102.

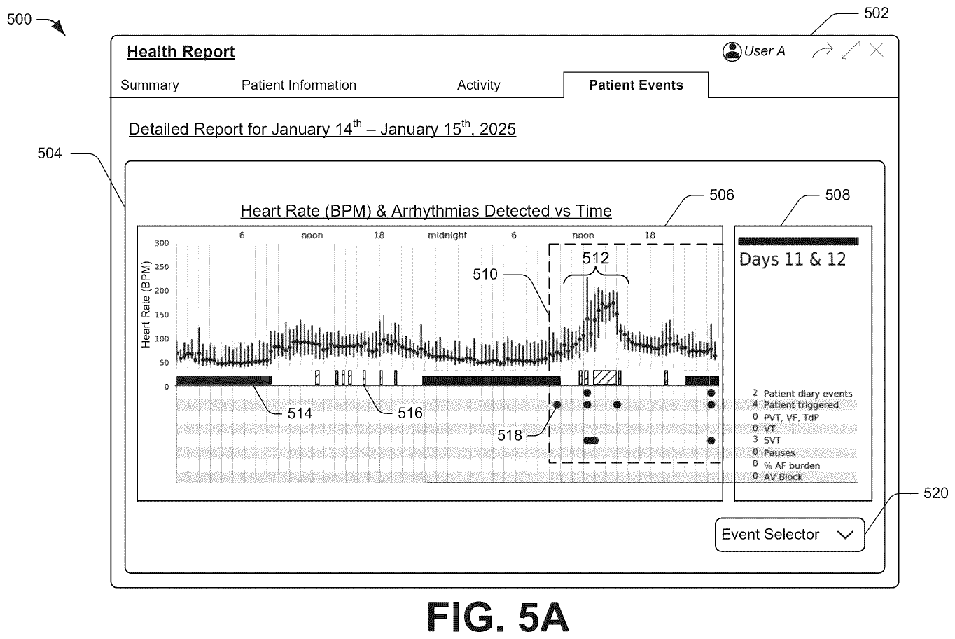

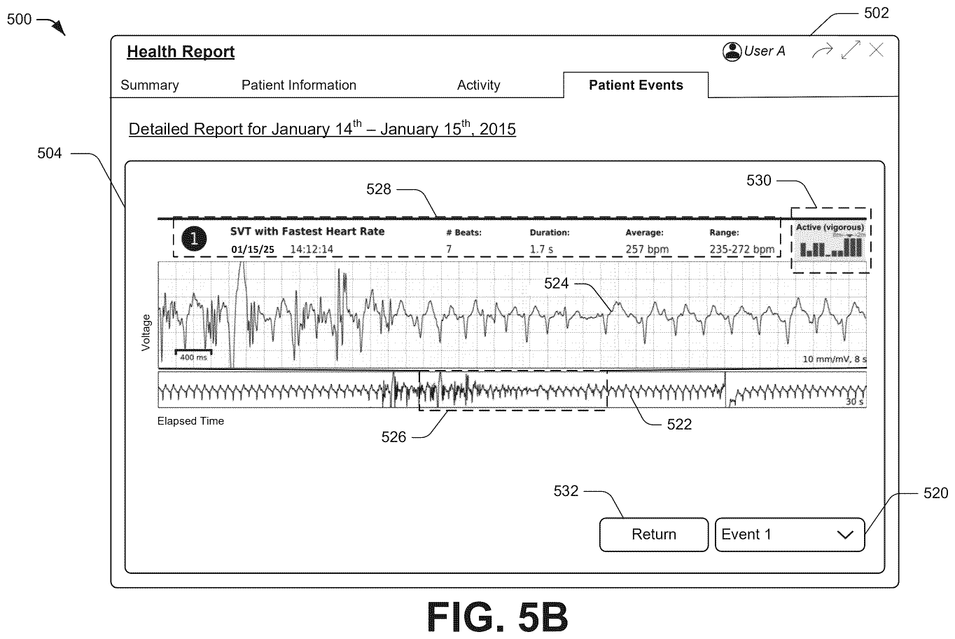

FIGS. 5A and 5B show a non-limiting example 500 of a user interface for displaying patient cardiac monitoring data with sleep and activity correlation. As depicted in the illustrated example 500, a user interface 502 presents comprehensive cardiac monitoring results that include various insights derived from the arrhythmia correlations 126, the sleep stage classifications 120, and the activity level classification 122 generated in accordance with the techniques described herein. The user interface 502 represents results from an observation period in which the person 102 wears the monitoring device 104 to capture the sensor data 302, including electrical data 304 and accelerometer data 306. In some examples, the user interface 502 displays data generated during the observation period. Additionally or alternatively, the user interface 502 displays data generated after the observation period has concluded.

In this example, the user interface 502 includes a patient events report section 504 that displays detailed cardiac monitoring information for a specific time period (e.g., Jan. 14th-15th, 2025). The patient events report section 504 provides a comprehensive view of cardiac events correlated with sleep and activity states detected by the cardiac monitoring system 118 and the activity detection system 116, respectively. A graph 506 within the patient events report section 504 displays heart rate measurements in beats per minute (BPM) and detected arrhythmias plotted against time over the specific time period. The graph 506 enables visualization of cardiac rhythm patterns and their temporal relationships with patient activities and sleep cycles.

An information panel 508 positioned adjacent to the graph 506 provides contextual details about the displayed data, such as indicating “Days 11 & 12” of the monitoring period. The information panel 508 may display statistical summaries, measurement parameters, or other relevant metadata associated with the cardiac monitoring session. In this example, the information panel 508 also labels specific events, e.g., patient diary events, patient triggered events, and specific cardiac events and/or anomalies. An event region 510 indicates a portion of the graph of interest where cardiac events or anomalies have been indicated, e.g., via the cardiac rhythm classification 124. Within the event region 510, an elevated heart rate region 512 indicates a period where the heart rate measurement exceeds normal baseline levels, which may correspond to arrhythmic episodes or increased cardiac activity.

The graph 506 further includes sleep regions 514 and active regions 516 displayed below the heart rate trace to provide activity context for the cardiac measurements. The sleep regions 514 indicate time periods when the person 102 was determined to be in a sleep state based on analysis of the accelerometer data 306 by the activity detection system 116. The active regions 516 indicate time periods when the person 102 was determined to be in an active state, such as periods involving movement equivalent to walking speed of 2 mph or greater as detected by the activity detection system 116. Event markers 518 are positioned throughout the graph 506 to denote specific patient events, such as arrhythmic episodes, symptomatic events, or other clinically relevant occurrences identified during the monitoring period. An event selector 520 positioned at the bottom of the patient events report section 504 enables navigation between different detected events and time periods within the monitoring data. The event selector 520 may allow users to select specific events marked by the event markers 518 for detailed examination, enabling transition to the expanded analysis view shown in FIG. 5B in response to the selection.

FIG. 5B shows additional details of the user interface 502 including expanded analysis capabilities for examining specific cardiac events accessed through the event selector 520 from FIG. 5A. By way of example, the event selector 520 shows “Event 1” selected in this example. A voltage plot 522 displays electrocardiogram waveform data corresponding to the selected cardiac event, showing the electrical potential measurements captured by the monitoring device 104 during the time period corresponding to that specific event. In the present example, “Event 1” is a supraventricular tachycardia (SVT) event where the heart rate is unusually fast and originates above the ventricles (e.g., in the atria or the atrioventricular node). The voltage plot 522 indicates the cardiac signal morphology that was analyzed to detect and classify the SVT (e.g., as a part of the cardiac rhythm classification 124).

A magnified voltage plot 524 provides an enlarged view of a selected portion of the voltage plot 522, enabling detailed examination of waveform characteristics such as ST segment changes, T wave morphology, or other abnormalities caused by the SVTs. An event region 526 on the voltage plot 522 corresponds to the portion included in the magnified voltage plot 524. In at least one implementation, the event region 526 may be adjusted in response to user input in order to display another portion of the voltage plot 522 in the magnified voltage plot 524.

An information panel 528 displays detailed metrics and contextual information about the selected cardiac event, including heart rate measurements, event duration, and timing information. The information panel 528 may present quantitative analysis results generated by the cardiac monitoring system 118, such as rhythm classifications, beat-to-beat intervals, or other cardiac parameters derived from the electrical data 304. An activity level indicator 530 shows the activity intensity level associated with the selected cardiac event, such as “Active (vigorous)” in this example. The activity intensity level may correspond to the activity level classification 122 determined by the activity detection system 116. The activity level indicator 530 provides context about the patient activity state during the occurrence of the cardiac event, enabling correlation between physical activity and arrhythmic episodes.

A return button 532 enables navigation back to the main view of the patient events report section 504 (e.g., FIG. 5A) from the detailed event analysis interface. The return button 532 facilitates user workflow by allowing transitions between overview and detailed analysis modes within the user interface 502. The comprehensive interface design enables healthcare providers to examine cardiac monitoring data at multiple levels of detail while maintaining awareness of sleep and activity context throughout the analysis process.

In some implementations, the user interface 502 may incorporate results from odds ratio and/or regression analysis performed on large patient populations to identify arrhythmia trends and patterns. Such population-level analysis enables the prediction system 114 to generate more accurate arrhythmia correlations 126 by leveraging statistical insights derived from extensive patient datasets. By way of example, the odds ratio analysis 420 may be provided within the user interface 502 to demonstrate statistically significant associations with specific sleep or activity states based on population-level trends.

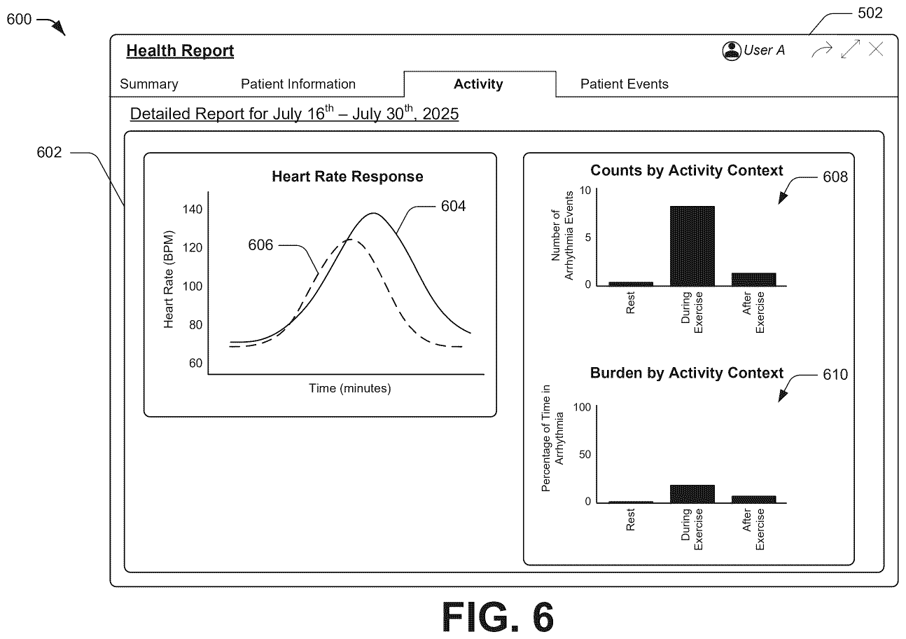

FIG. 6 shows an example 600 of a comprehensive activity-based cardiac assessment interface. The example 600 is another example of the user interface 502 introduced in FIG. 5A. In this example 600, the user interface 502 displays an activity report section 602 that provides a detailed analysis of cardiac performance during various exercise periods. The activity report section 602 includes multiple graphical representations that enable healthcare providers to evaluate cardiac response patterns and identify exercise-related arrhythmias over extended monitoring periods.

The activity report section 602 includes a heart rate response curve 604 that displays actual heart rate measurements recorded during detected activity events. The heart rate response curve 604 presents time series data showing heart rate data collected from the onset of physical activity through a recovery period. An expected heart rate curve 606 overlays the heart rate response curve 604 to provide comparative analysis between predicted and observed cardiac responses. The expected heart rate curve 606 may be generated using algorithmic models that account for patient demographics, baseline fitness levels, and established cardiac response parameters, for instance. The comparison between the heart rate response curve 604 and the expected heart rate curve 606 enables detection of chronotropic incompetence, where cardiac output fails to increase appropriately during physical exertion. By way of example, the prediction system 114 may indicate chronotropic incompetence in response to the heart rate response curve 604 deviating from the expected heart rate curve 606 by at least a threshold amount.

An activity context graph 608 displays a quantitative analysis of arrhythmia events across different activity states. The activity context graph 608 categorizes cardiac events into distinct phases including rest periods (e.g., “rest”), active exercise periods (e.g., “during exercise”), and post-exercise recovery periods that immediate follow the active exercise periods (e.g., “after exercise”). The activity context graph 608 shows event counts for each activity context, allowing healthcare providers to identify patterns where arrhythmias occur more frequently during specific activity levels. In at least one variation, the activity context graph 608 may separate data by activity intensity levels, including light, moderate, and vigorous exercise categories based on movement patterns equivalent to walking speeds and accelerometer-derived activity classifications (e.g., the activity level classification 122).

The activity report section 602 further includes an arrhythmia burden graph 610 that quantifies the percentage of time spent in various arrhythmic states during different activity contexts. The arrhythmia burden graph 610 may present data as percentage values, allowing for standardized comparison across different monitoring periods and patient populations. The activity report section 602 may thus display a comprehensive assessment of exercise-related cardiac evaluation.

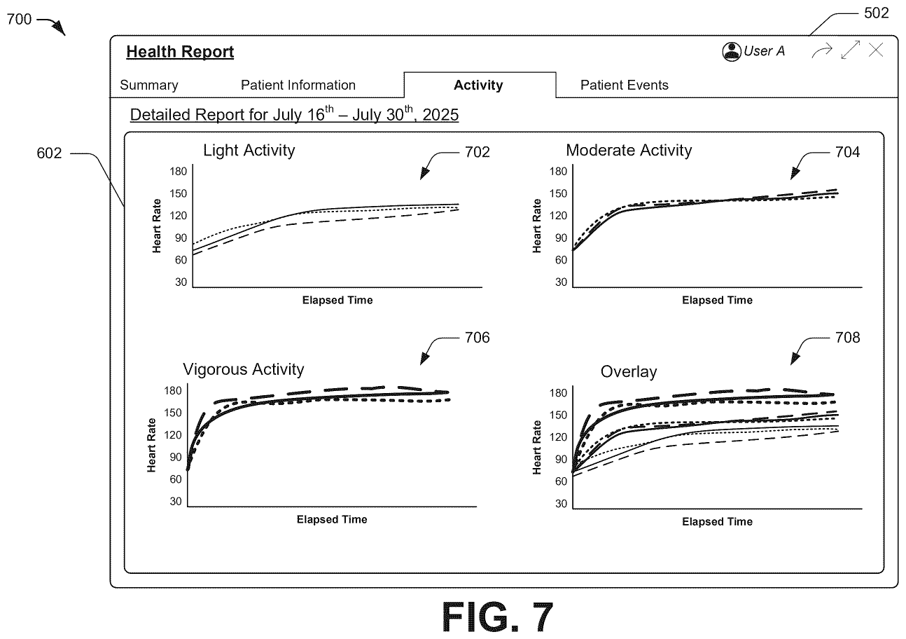

FIG. 7 shows an example 700 of a multi-level activity classification display that provides comprehensive analysis of cardiac activity across different exercise intensities. The example 700 shows another example of the activity report section 602. In this example, the activity report section 602 includes multiple graphical representations for analyzing heart rate responses during various activity levels over an extended monitoring period. By way of example, the activity report section 602 may include heart rate response stratified by activity level.

The activity report section 602 includes a light activity graph 702, a moderate activity graph 704, a vigorous activity graph 706, and an overlay graph 708. The light activity graph 702 displays heart rate measurements collected during periods of light physical activity, such as walking or gentle movement. The moderate activity graph 704 presents heart rate data collected during moderate intensity activities, which may include activities equivalent to brisk walking or light jogging. The moderate activity graph 704 may demonstrate increased heart rate responses compared to the light activity periods. The vigorous activity graph 706 displays heart rate measurements during high-intensity physical activities, such as running, climbing stairs, or other strenuous exercises. Each of the light activity graph 702, moderate activity graph 704, and vigorous activity graph 706 presents heart rate data plotted against elapsed time, allowing for detailed analysis of cardiac responses within specific activity intensity categories. Multiple plots may be displayed on each graph, with different line patterns (e.g., solid, short-dashed, long-dashed) indicating data obtained during a different activity event. Moreover, different line widths are used in this example to differentiate between different activity levels, with the light activity graph 702 including the thinnest lines and the vigorous activity graph 706 including the thickest lines. In variations, other visual indicators (such as color, labels, or other line styles), may be used to distinguish the different plots from one another.

The overlay graph 708 combines heart rate data from all activity intensity levels to provide a comprehensive view of cardiac performance across the spectrum of physical activities. This combined visualization enables healthcare providers to compare heart rate responses across different exercise intensities and identify patterns or abnormalities that may occur during specific activity levels. The overlay graph 708 may facilitate detection of chronotropic incompetence, which may not be apparent during rest periods or single-intensity monitoring.

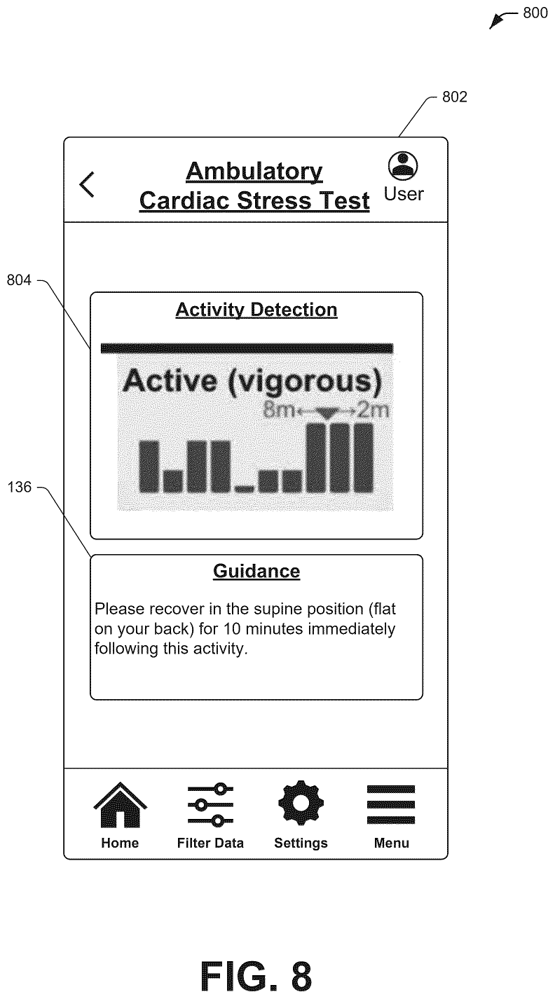

FIG. 8 shows an example 800 of an ambulatory cardiac stress test interface that facilitates monitoring and analysis of cardiac activity during extended wear periods. The example 800 includes a user interface 802, which provides navigation and control functionality to the person 102 to employ an ambulatory cardiac stress test procedure.

An activity graph 804 displays vigorous activity detection data, showing periods of intense physical activity that may trigger cardiac stress responses during the extended monitoring period. The activity graph 804 presents temporal data indicating that vigorous activity has been detected.

The user interface 802 displays the instructions 136, which provide specific guidance for post-exercise recovery positioning to optimize cardiac data collection for ambulatory cardiac stress testing in this example. The instructions 136 direct the person 102 to recover in the supine position for 10 minutes immediately following the detected vigorous activity, which enables collection of electrical data 304 in the supine position in order to gather specific data that may be used for the cardiac stress test. The instructions 136 may be automatically triggered when the monitoring device 104 detects vigorous activity through analysis of the accelerometer data 306, ensuring that recovery positioning occurs at appropriate times during the extended wear period.

The user interface 802 supports detection of cardiac abnormalities that may manifest during or after exercise periods, including ST segment depression, ST segment elevation, T wave inversion, T wave flattening, and pseudonormalization of T waves, by issuing the instructions 136 to facilitate the collection of data in appropriate activity and rest states. The monitoring device 104 may thus be used to acquire diagnostic information comparable to traditional exercise stress testing while allowing for extended monitoring in ambulatory settings.

The following section describes example procedures for a cardiac monitoring system with sleep and activity correlation in one or more implementations. Aspects of each of the procedures can be implemented in hardware, firmware, software, or a combination thereof. The procedures are shown as a set of blocks that specify operations that can be performed by one or more devices and are not necessarily limited to the orders shown for performing the operations by the respective blocks. One or more blocks of the procedures, for instance, specify operations that can be programmable by hardware (e.g., processor, microprocessor, controller, firmware) as instructions thereby creating a special purpose machine for carrying out an algorithm as illustrated by the flow diagram. As a result, the instructions are storable on a computer-readable storage medium that causes the hardware to perform the algorithm. In portions of the following discussion, reference will be made to FIGS. 1-8.

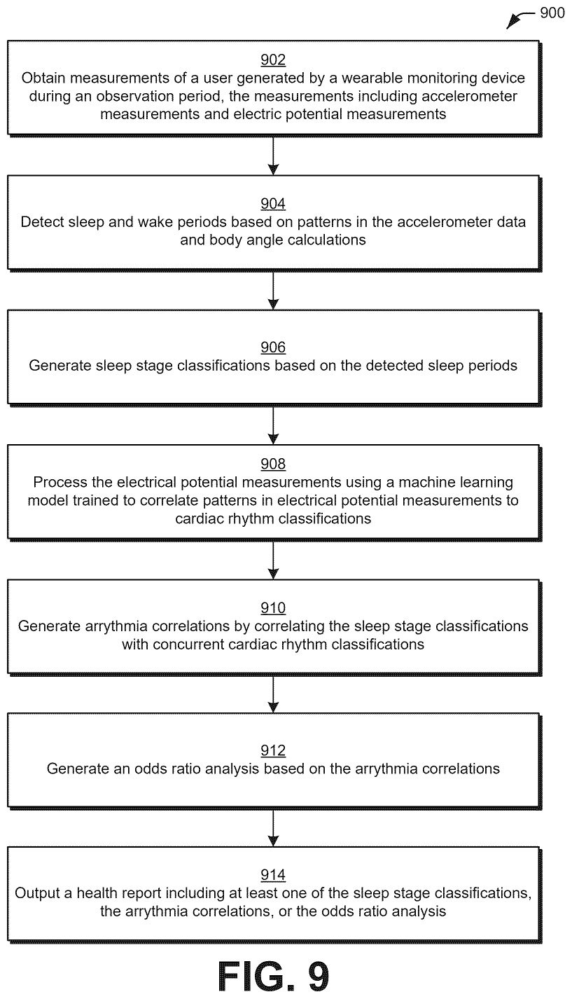

FIG. 9 depicts a flow diagram depicting an algorithm as a step-by-step procedure 900 in an example implementation that is performable by a processing device to correlate cardiac monitoring with sleep and activity data based on measurements from a wearable monitoring device. The procedure 900 processes both accelerometer measurements and electric potential measurements to produce analyses correlating arrhythmia episodes with sleep and/or activity periods.

To begin in this example, measurements of a user are obtained by a wearable monitoring device during an observation period, the measurements including accelerometer measurements and electric potential measurements (block 902). The measurements 108, for instance, are produced by the monitoring device 104 during continuous wear by the person 102. In various examples, the monitoring device 104 detects movement patterns based on the accelerometer data 306 while simultaneously capturing electrical activity of the heart using the one or more sensors 202. The accelerometer data 306 may be sampled at frequencies lower than conventional activity trackers, which typically operate at 25-100 Hz, thereby conserving battery life and memory resources of the monitoring device 104. The electrical data 304 (e.g., electrical potential measurements) may be collected substantially continuously to provide cardiac rhythm information that can be correlated with detected movement patterns.

The monitoring device 104 may at least partially process the measurements 108 locally, before transmission to the analysis platform 106. Alternatively, the analysis platform 106 may be included as part of the monitoring device 104. In yet another example, the monitoring device 104 may transmit the measurements 108 to the analysis platform 106 for processing. Additionally or alternatively, the monitoring device 104 may compress the sensor data using various data compression techniques to reduce battery usage during transmission (e.g., wireless or wired transmission) to external computing devices after the observation period concludes.

Sleep and wake periods are detected based on patterns in the accelerometer data and body angle calculations (block 904). The activity detection system 116, for instance, processes the accelerometer measurements (e.g., the accelerometer data 306) to identify periods of stillness and periods of movement that correspond to sleep and wake cycles. Body angle calculations are performed using the accelerometer measurements to determine when the user is in reclined positions that indicate sleep periods versus upright positions that indicate wake periods. The activity detection system 116 may analyze movement patterns, body positioning, and activity levels (e.g., based on step counts within a predetermined time epoch) to distinguish between different physiological periods throughout the observation period. The activity detection system 116 may analyze movement patterns, body positioning, and activity levels (e.g., based on step counts within a predetermined time epoch) to distinguish between different physiological periods throughout the observation period. The activity detection system 116 may classify sleep based on stillness detected through body motion and body angle analysis, while wake periods are identified through movement patterns and upright positioning.

Sleep stage classifications are generated based on the detected sleep periods (block 906). The prediction system 114, for instance, processes the detected sleep periods to generate the sleep stage classifications 120, which may categorize different phases of sleep, such as light sleep, deep sleep, REM sleep, and/or non-REM sleep. The sleep stage classifications 120 may provide a temporal mapping of when the user experiences different sleep phases.

The electrical potential measurements are processed using a machine learning model trained to correlate patterns in electrical potential measurements to cardiac rhythm classifications (block 908). The trained machine learning model 326, for instance, receives the electrical potential measurements as input and analyzes features such as heart rate variability, rhythm patterns, and electrical signal morphology to classify different types of arrhythmias and cardiac events, e.g., the cardiac rhythm classifications 124. The trained machine learning model 326 is trained using historical electrical potential measurements and historical outcome data of a user population to perform cardiac rhythm classification tasks. The cardiac rhythm classification 124 identifies specific types of arrhythmias, heart rate variations, and other cardiac events that occur at specific times during the observation period.

Arrhythmia correlations are generated by correlating the sleep stage classifications with concurrent cardiac rhythm classifications (block 910). The prediction system 114, for instance, performs temporal alignment of the sleep stage classification 120 with the cardiac rhythm classification 124 to identify relationships between sleep periods and arrhythmia occurrences, e.g., via the aggregator 416. The arrhythmia correlations 126 indicate which specific types of cardiac events occur relative to different sleep stages, providing insights into whether arrhythmias are more likely during specific sleep stages.

An odds ratio analysis is generated based on the arrhythmia correlations (block 912). The prediction system 114, for instance, performs statistical analysis on the correlated data to calculate odds ratios that quantify the likelihood of specific arrhythmias occurring during different sleep stages. The odds ratio analysis 420 provides quantitative measures of association between sleep stages and arrhythmia occurrence, enabling healthcare providers to understand the statistical significance of observed patterns. The analysis may include calculations comparing arrhythmia rates during sleep versus wake periods, for instance.

A health report is output including at least one of the sleep stage classifications, the arrhythmia correlations, or the odds ratio analysis (block 914). The health report 418, for instance, presents a comprehensive analysis in a format suitable for clinical review and decision-making. The report may include visualizations that overlay sleep and activity information with cardiac rhythm events, providing healthcare providers with intuitive representations of the arrhythmia correlations 126. In various examples, the health report 418 includes percentage breakdowns of arrhythmia types during different sleep stages, statistical analysis results, and temporal patterns that assist in diagnosis and treatment planning.

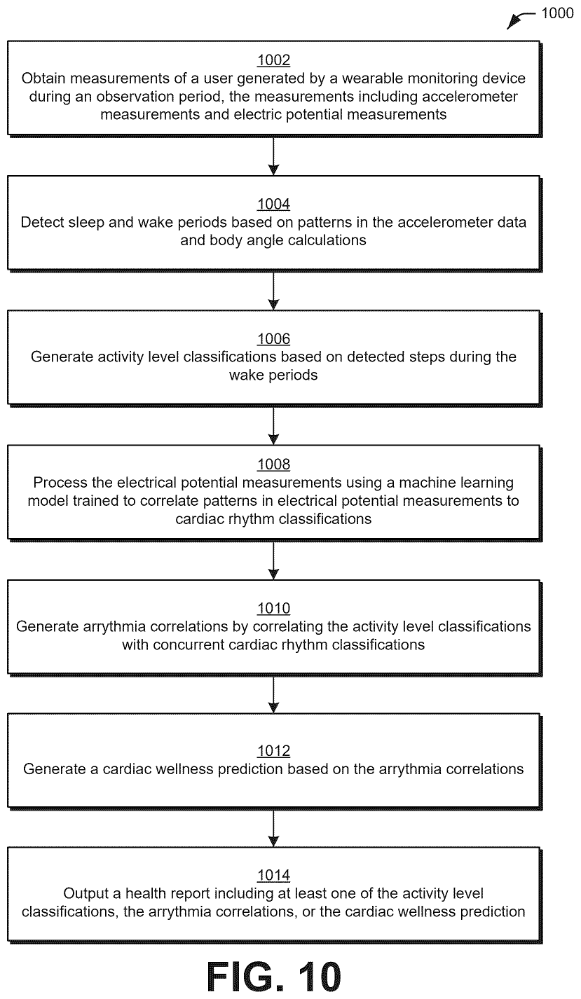

FIG. 10 depicts a flow diagram depicting an algorithm as a step-by-step procedure 1000 in an example implementation that is performable by a processing device to perform cardiac monitoring with activity level classification and arrhythmia correlation. The procedure 1000 enables comprehensive analysis of cardiac events in relation to physical activity states, providing healthcare providers with detailed insights into exercise-related cardiac patterns and wellness predictions.

Measurements of a user are obtained by a wearable monitoring device during an observation period, the measurements including accelerometer measurements and electric potential measurements (block 1002). By way of example, the monitoring device 104 continuously collects physiological data 302 from the person 102 over an extended monitoring period. The accelerometer data 306 captures movement patterns and physical activity levels, while the electrical data 304 records cardiac electrical activity through the one or more sensors 202. The monitoring device 104 may operate at a low sample rate of less than 5 Hz (e.g., 1.6 or 1.56 Hz) for the accelerometer data 306 to conserve battery power and memory resources during the observation period. The extended wear period may range between 7 and 14 days to capture comprehensive activity patterns and cardiac events across multiple daily cycles. Additional example details are described herein, e.g., with respect to block 902 of FIG. 9.

Sleep and wake periods are detected based on patterns in the accelerometer data and body angle calculations (block 1004). The activity detection system 116, for instance, processes the accelerometer measurements (e.g., the accelerometer data 306) to identify periods of stillness and periods of movement that correspond to sleep and wake cycles. Body angle calculations are performed using the accelerometer measurements to determine when the user is in reclined positions that indicate sleep periods versus upright positions that indicate wake periods. The activity detection system 116 may analyze movement patterns, body positioning, and activity levels (e.g., based on step counts within a predetermined time epoch) to distinguish between different physiological periods throughout the observation period. The activity detection system 116 may classify sleep based on stillness detected through body motion and body angle analysis, while wake periods are identified through movement patterns and upright positioning.

Activity level classifications are generated based on detected steps during the wake periods (block 1006). By way of example, the activity detection system 116 performs step counting to determine the activity level classification 122 during periods when the person 102 is determined to be awake. The activity detection system 116 may classify activity (versus inactivity) based on movement equivalent to walking speed of 2 mph or greater. The activity may be further categorized into light, moderate, and vigorous intensity levels based on the number of steps in predetermined time epochs according to a plurality of step thresholds. By way of example, the activity detection system 116 may process the accelerometer data 306 to detect steps as peaks above a measurement threshold. In one or more implementations, the monitoring device 104 may include a gyroscope that can be triggered when activity is detected to provide additional insight into activity type or intensity. In some such examples, the gyroscope may remain inactive during periods of inactivity to conserve power.

The electrical potential measurements are processed using a machine learning model trained to correlate patterns in electrical potential measurements to cardiac rhythm classifications (block 1008). The trained machine learning model 326, for instance, receives the electrical potential measurements as input and analyzes features such as heart rate variability, rhythm patterns, and electrical signal morphology to classify different types of arrhythmias and cardiac events, e.g., the cardiac rhythm classifications 124. The trained machine learning model 326 is trained using historical electrical potential measurements and historical outcome data of a user population to perform cardiac rhythm classification tasks. The cardiac rhythm classification 124 identifies specific types of arrhythmias, heart rate variations, and other cardiac events that occur at specific times during the observation period.

Arrhythmia correlations are generated by correlating the activity level classifications with concurrent cardiac rhythm classifications (block 1010). The prediction system 114, for instance, performs temporal alignment of the activity level classification 122 with the cardiac rhythm classification 124 to identify relationships between activity periods and arrhythmia occurrences, e.g., via the aggregator 416. The arrhythmia correlations 126 indicate which specific types of cardiac events occur relative to different activity levels, providing insights into whether arrhythmias are more likely before, during, or after activities of certain intensity levels.

A cardiac wellness prediction is generated based on the arrhythmia correlations (block 1012). By way of example, the prediction system 114 processes the arrhythmia correlation 126 results to generate the cardiac wellness prediction 128, which may provide insights into overall cardiac health status. The prediction system 114 may detect chronotropic incompetence by comparing expected and actual heart rate responses during detected activity events, identifying cases where heart rate does not increase as expected during exercise. The cardiac wellness prediction 128 may incorporate analysis of heart rate variability patterns, recovery rates following exercise periods, and/or the frequency and timing of arrhythmia episodes relative to activity levels. In one or more implementations, the cardiac wellness prediction 128 may compare rhythm burdens and counts during exercise periods against non-exercise wake periods. The cardiac wellness prediction 128 may also account for patterns observed across multiple activity sessions captured during the extended monitoring period.

A health report is output including at least one of the activity level classifications, the arrhythmia correlations, or the cardiac wellness prediction (block 1014). By way of example, the prediction system 114 generates the health report 418, which may present comprehensive analysis results through visualizations and quantitative summaries. The health report 418 may include the activity level classification 122 data showing daily activity patterns, step counts, and/or intensity level distributions across the monitoring period. The arrhythmia correlation 126 results may be presented as overlays of cardiac events with activity periods, providing healthcare providers with context for when specific arrhythmias occurred relative to physical activity. The cardiac wellness prediction 128 may be incorporated into the health report 418 as recommendations or assessments regarding cardiac fitness and exercise tolerance based on the observed correlations between activity and cardiac response patterns.

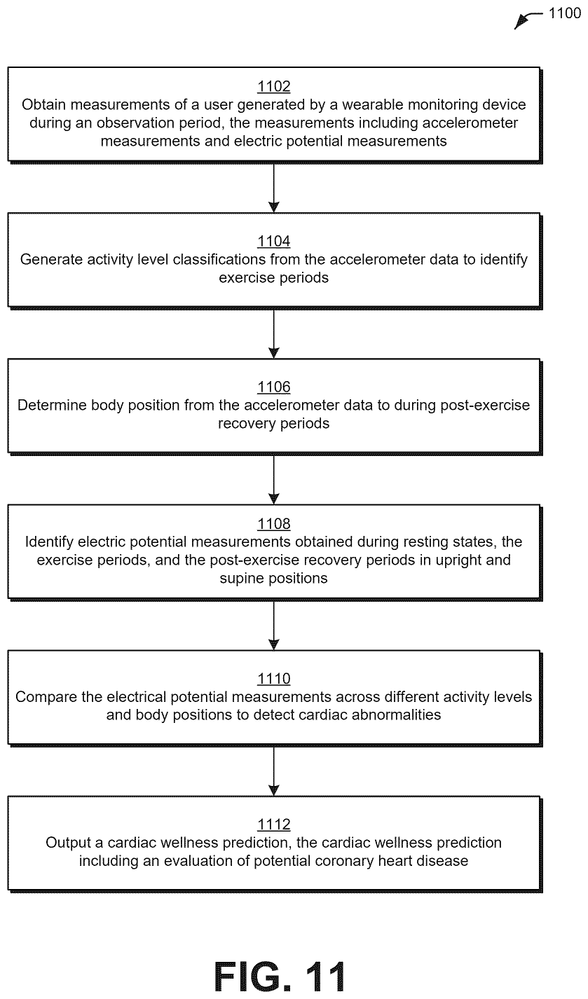

FIG. 11 depicts a flow diagram depicting an algorithm as a step-by-step procedure 1100 in which ambulatory cardiac stress testing is performed using a wearable monitoring device.

Measurements of a user are obtained from a wearable monitoring device during an observation period, the measurements including accelerometer measurements and electric potential measurements (block 1102). By way of example, the monitoring device 104 continuously collects the measurements 108 over an extended wear period that may span between 7 and 14 days. The accelerometer data 306 captures movement patterns and body positioning information at a low sample rate (e.g., less than 5 Hz) to conserve power and memory resources of the monitoring device 104. The electrical data 304 may represent continuous electrocardiogram signals that record the electrical activity of the heart throughout the observation period. The extended monitoring duration enables capture of multiple exercise events and recovery periods that occur during normal daily activities, providing a comprehensive dataset for ambulatory stress testing analysis. Additional details are described herein, e.g., at block 902 of FIG. 9 and block 1002 of FIG. 10.

Activity level classifications are generated from the accelerometer data to identify exercise periods (block 1104). By way of example, the activity detection system 116 processes the accelerometer data 306 to detect movement patterns equivalent to active states indicative of exercise periods. As a non-limiting example, exercise periods may correspond to walking speeds of 2 mph or greater. The prediction system 114 may classify the activity level according to light, moderate, and vigorous categories based on the magnitude and frequency of detected movements and/or based on step counts within predetermined time epochs. The activity level classification 122 may identify discrete exercise events that can be used for stress testing analysis, including the onset and duration of each activity period. The system may also detect when a gyroscope should be activated to provide additional insight into the type or intensity of the detected activity.

Body position is determined from the accelerometer data during post-exercise recovery periods (block 1106). By way of example, the activity detection system 116 calculates body angle within a 0-90 degree range from upright to recumbent position using the accelerometer data 306. The activity detection system 116 may compute a reference vector based on high activity regions where the patient may be in an upright position and then determine the body angle at each time point as the polar angle against the reference upright vector. During recovery phases following exercise, the activity detection system 116 may identify when the person 102 transitions to a supine position, which may precipitate ischemic abnormalities not visible during exercise. The body position determination enables the prediction system 114 to categorize electrical potential measurements based on whether the patient was upright or supine during data collection. The accelerometer data 306 may thus provide continuous monitoring of body positioning changes throughout the recovery period without relying on patient input or manual positioning protocols.

Electric potential measurements obtained during resting states, the exercise periods, and the post-exercise recovery periods in upright and supine positions are identified (block 1108). By way of example, the cardiac monitoring system 118 processes the electrical data 304 to extract electrocardiogram segments corresponding to different physiological states and body positions. The cardiac monitoring system 118 may identify resting electrocardiogram data collected when the patient was in both standing and supine positions before exercise events. During exercise periods identified by the activity level classification 122, the cardiac monitoring system 118 may identify electrocardiogram data acquired while the patient was in an upright position performing physical activities. The cardiac monitoring system 118 may further identify electrocardiogram data acquired during recovery periods, such as electric potential measurements obtained each minute for up to 10 minutes after exercise when the person 102 was in a supine position. The feature extraction process 412 may organize the electrical potential measurements according to the concurrent activity state and body position to enable systematic comparison across different physiological conditions.

The electrical potential measurements are compared across different activity levels and body positions to detect cardiac abnormalities (block 1110). By way of example, the cardiac monitoring system 118 may analyze electrocardiogram patterns to identify abnormalities indicative of myocardial ischemia, which represents a mismatch between myocardial oxygen delivery and myocardial oxygen demand. The cardiac monitoring system 118 may detect ST segment depression, ST segment elevation, T wave inversion, T wave flattening, and pseudonormalization of T waves by comparing exercise and recovery electrocardiogram data to resting baseline measurements collected in the same body positions. In one or more implementations, the trained machine learning model 326 processes the electrical potential measurements to identify patterns associated with coronary artery disease that may manifest during exercise-induced stress or during post-exercise recovery phases. The comparison process accounts for the different body positions during data collection, as electrocardiogram morphology can vary between upright and supine positions independent of cardiac pathology.

A cardiac wellness prediction is output, the cardiac wellness prediction including an evaluation of potential coronary heart disease (block 1112). By way of example, the prediction system 114 generates the cardiac wellness prediction 128 based on the analysis of electrocardiogram abnormalities detected during the ambulatory cardiac stress testing procedure. The cardiac wellness prediction 128, or the health report 418 generated therefrom, may include a panel of sample electrocardiograms from each physiological condition, including resting electrocardiogram in supine position, resting electrocardiogram in upright position, upright position electrocardiogram each minute after exercise for up to 10 minutes, and supine position electrocardiogram each minute after exercise for up to 10 minutes. The cardiac wellness prediction 128 may provide an assessment of coronary heart disease risk based on the comprehensive analysis of cardiac function during real-world exercise conditions rather than controlled laboratory protocols. The user interface 502 may display the cardiac wellness prediction 128 along with supporting electrocardiogram data and activity context to enable clinical interpretation of the ambulatory stress test results.

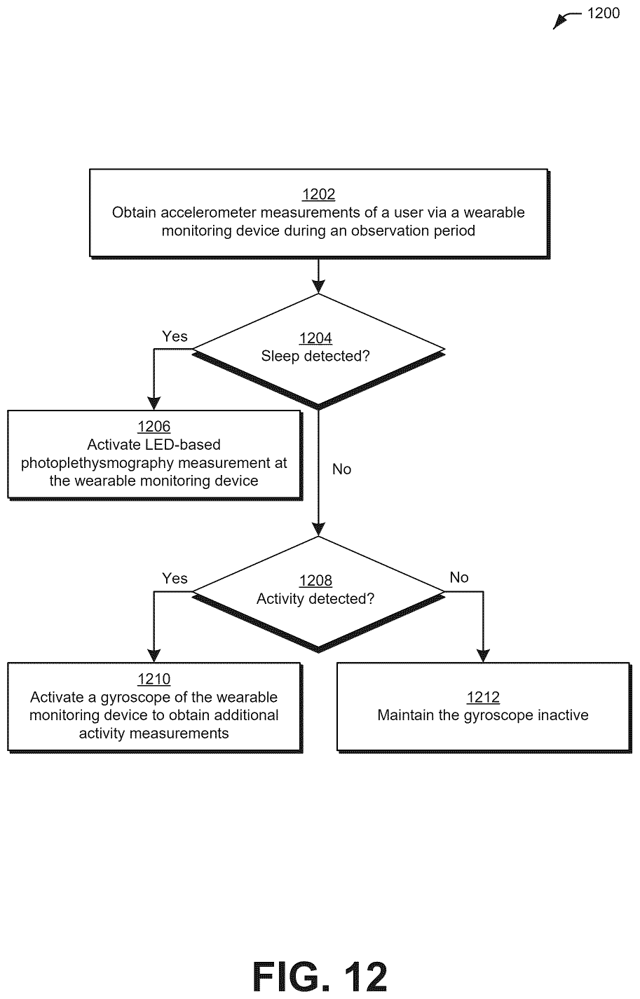

FIG. 12 depicts a flow diagram depicting an algorithm as a step-by-step procedure 1200 in which sleep/activity triggering of analysis and data acquisition is performed.

Accelerometer measurements of a user are obtained via a wearable monitoring device during an observation period (block 1202). By way of example, the monitoring device 104 continuously collects accelerometer data 306 to detect movement patterns and body positioning information throughout the observation period. The accelerometer data 306 may be sampled at a low rate (e.g., less than 5 Hz) to conserve battery power while maintaining accurate detection capabilities for sleep and activity pattern recognition.

It is determined if sleep is detected based on the accelerometer measurements (block 1204). By way of example, the activity detection system 116 processes the accelerometer data 306 to identify periods of stillness and body positioning that correspond to sleep states. The sleep wake analysis 402 may include analyzing body angle measurements and movement patterns to distinguish between sleep and wake periods, for instance.

If sleep is detected (e.g., “yes” at block 1204), LED-based photoplethysmography measurement is activated at the wearable monitoring device (block 1206). By way of example, the monitoring device 104 may trigger the more battery-intensive data acquisition of photoplethysmography for SpO2 data 308 collection, which may be used for sleep apnea screening and diagnosis. The LED-based photoplethysmography may turn off when the person 102 is no longer asleep to conserve battery resources during the observation period.

If sleep is not detected (e.g., “no” at block 1204), it is determined if activity is detected based on the accelerometer measurements (block 1208). By way of example, the activity detection system 116 analyzes the accelerometer data 306 to identify movement patterns equivalent to walking speeds greater than a threshold (e.g., 2 mph) to distinguish between active and inactive awake states.

If activity is detected (e.g., “yes” at block 1208), a gyroscope of the wearable monitoring device is activated to obtain additional activity measurements (block 1210). By way of example, when activity is detected by the activity detection system 116, the monitoring device 104 may turn on additional channels of data acquisition such as the gyroscope, which are more costly in terms of battery consumption but may provide additional insight into the type or intensity of the activity performed. The gyroscope activation enables enhanced activity classification and correlation with concurrent cardiac measurements from the electrical data 304, for instance.

If activity is not detected (e.g., “no” at block 1208), the gyroscope remains inactive (block 1212). By way of example, the monitoring device 104 may maintain standard monitoring parameters without activating power-intensive sensors when the person 102 is in an inactive wake state. The selective sensor activation approach increases battery life while ensuring comprehensive data capture during physiologically relevant periods for cardiac wellness prediction 128 and arrhythmia correlation 126 analysis, for instance.

The various functional units illustrated in the figures and/or described herein are implemented in any of a variety of different manners such as hardware circuitry, software or firmware executing on a programmable processor, or any combination of two or more of hardware, software, and firmware. The methods provided are implemented in any of a variety of devices, such as a general-purpose computer, a processor, or a processor core. Suitable processors include, by way of example, a general purpose processor, a special purpose processor, a conventional processor, a digital signal processor (DSP), a graphics processing unit (GPU), a parallel accelerated processor, a plurality of microprocessors, one or more microprocessors in association with a DSP core, a controller, a microcontroller, Application Specific Integrated Circuits (ASICs), Field Programmable Gate Arrays (FPGAs) circuits, any other type of integrated circuit (IC), and/or a state machine.

In one or more implementations, the methods and procedures provided herein are implemented in a computer program, software, or firmware incorporated in a non-transitory computer-readable storage medium for execution by a general-purpose computer or a processor. Examples of non-transitory computer-readable storage mediums include a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

It should be understood that many variations are possible based on the disclosure herein. Although features and elements are described above in particular combinations, each feature or element is usable alone without the other features and elements or in various combinations with or without other features and elements.

What is claimed is:

1. A method implemented by a processing device, the method comprising:
obtaining measurements of a user generated by a wearable monitoring device during an observation period, the measurements including accelerometer data and electrical potential measurements;
detecting sleep periods and wake periods based on the accelerometer data;
generating sleep stage classifications based on the accelerometer data during the detected sleep periods;
processing the electrical potential measurements using a machine learning model trained to correlate patterns in the electrical potential measurements to cardiac rhythm classifications;
generate arrhythmia correlations based on the sleep stage classifications and concurrent cardiac rhythm classifications, the arrhythmia correlations describing temporal relationships between detected cardiac arrhythmias and specific sleep stages of the sleep stage classifications; and
outputting a health report including the arrhythmia correlations.

2. The method of claim 1, wherein the accelerometer data is collected at a sampling rate of less than 5 Hertz.

3. The method of claim 1, wherein detecting the sleep periods and the wake periods based on the accelerometer data comprises:
detecting steps as peaks above a threshold in the accelerometer data;
calculating body angle measurements from the accelerometer data to determine body positioning; and
distinguishing between the sleep periods and the wake periods based on the detected steps and the body angle measurements.

4. The method of claim 1, wherein generating the sleep stage classifications comprises categorizing the sleep periods as light sleep, deep sleep, rapid eye movement (REM) sleep, and non-REM sleep.

5. The method of claim 1, further comprising:
indicating activity events during the wake periods based on the accelerometer data obtained during the wake periods;
generating activity level classifications of the activity events based on an intensity of physical activity performed; and
generating additional arrhythmia correlations based on the activity level classifications and corresponding concurrent cardiac rhythm classifications, the additional arrhythmia correlations describing temporal relationships between the detected cardiac arrhythmias and specific activity levels of the activity level classifications.

6. The method of claim 5, wherein the activity level classifications categorize the physical activity as light, moderate, or vigorous based on movement equivalent to at least a threshold speed, as determined based on the accelerometer data.

7. The method of claim 5, further comprising:
detecting chronotropic incompetence based on an expected heart rate response relative to an actual heart rate response during the physical activity.

8. The method of claim 1, further comprising generating an odds ratio analysis based on the arrhythmia correlations, the odds ratio analysis quantifying a likelihood of specific arrhythmias occurring during the sleep periods versus the wake periods.

9. The method of claim 8, wherein the odds ratio analysis calculates statistical associations between the specific arrhythmias and the sleep periods across a user population.

10. The method of claim 1, wherein the health report includes a visualization of the arrhythmia correlations with the sleep periods and the wake periods.

11. A processing device, comprising:
one or more processors; and
memory having stored computer-readable instructions that are executable by the one or more processors to perform operations comprising:
obtaining measurements of a user generated by a wearable monitoring device during an observation period, the measurements including accelerometer data and electrical potential measurements;
detecting sleep periods and wake periods based on the accelerometer data;
generating sleep stage classifications based on the accelerometer data during the sleep periods;
generating activity level classifications based on detected steps during the wake periods;
processing the electrical potential measurements using a machine learning model trained to correlate patterns in the electrical potential measurements to cardiac rhythm classifications;
correlating the sleep stage classifications and the activity level classifications with concurrent cardiac rhythm classifications to generate arrhythmia correlations;
generating a cardiac wellness prediction based on the arrhythmia correlations; and
outputting a health report including the cardiac wellness prediction.

12. The processing device of claim 11, wherein the accelerometer data is collected at a sampling rate of less than 5 Hertz.

13. The processing device of claim 11, wherein generating the activity level classifications comprises categorizing physical activity as sedentary, light, moderate, or vigorous based on movement speed, as determined based on the accelerometer data.

14. The processing device of claim 13, wherein the operations further comprise:
detecting chronotropic incompetence by comparing an expected heart rate response and an actual heart rate response of the user during activity events within the wake periods.

15. The processing device of claim 14, wherein the operations further comprise:
determining a heart rate of the user over time based on the electrical potential measurements;
generating time series plots of the heart rate during detected the detected activity events;
stratifying the time series plots based on the activity level classifications; and
outputting the stratified time series plots as a part of the health report.

16. A system comprising:
a wearable monitoring device that is wearable by a user to detect one or more measurements of the user during an observation period, the one or more measurements including accelerometer measurements and electrical potential measurements of a heart of the user; and
a computing device configured to:
receive the one or more measurements from the wearable monitoring device;
identify exercise periods performed by the user during the observation period based on the accelerometer measurements;
process the electrical potential measurements using a machine learning model trained to correlate patterns in the electrical potential measurements to cardiac rhythm classifications;
generate arrhythmia correlations for periods before, during, and after the exercise periods based on concurrent cardiac rhythm classifications; and
generate a cardiac wellness prediction based on the arrhythmia correlations.

17. The system of claim 16, wherein the accelerometer measurements are collected at a sampling rate of less than 5 Hertz, and wherein the observation period is between 7 and 14 days.

18. The system of claim 16, wherein the computing device is further configured to:
generate an expected heart rate response during a given exercise period based on user population data;
generate an actual heart rate response of the user during the given exercise period based on the electrical potential measurements obtained during the given exercise period; and
indicate chronotropic incompetence based on the actual heart rate response deviating from the expected heart rate response by at least a threshold amount.

19. The system of claim 16, wherein to identify the exercise periods based on the accelerometer measurements, the computing device is further configured to:
detect steps as peaks in the accelerometer measurements above a measurement threshold; and
identify the exercise periods based on the detected steps exceeding a step count threshold during a predetermined time epoch.

20. The system of claim 16, wherein the computing device is further configured to:
detect sleep periods of the user based on the accelerometer measurements;
generate sleep stage classifications based on the accelerometer measurements during the detected sleep periods; and
generate additional arrhythmia correlations based on the sleep stage classifications and the concurrent cardiac rhythm classifications.
